(19) United States
(12) Patent Application Publication
(10) Pub. No.: US 2010/0240115 A1
(43) Pub. Date: Sep. 23, 2010

(54) BIOANALYTICAL ASSAY

(76) Inventor: Kim PETTERSSON, Turku (FI)

Correspondence Address: JAMES C. LYDON, 100 DAINGERFIELD ROAD, SUITE 100, ALEXANDRIA, VA, 22314, US

(21) Appl. No.: 12/636,642

(22) Filed: Dec. 11, 2009

Related U.S. Application Data
(63) Continuation of application No. 10/433,230, filed on Oct. 29, 2003, now abandoned.

(30) Foreign Application Priority Data
Nov. 30, 2000 (FI) 20002623

(51) Int. Cl. C12N 1/20; B32B 9/00

(52) U.S. Cl. 435/252.1; 428/402

(57) ABSTRACT

A nanoparticle having a detectable feature and whose diameter is less than 200 nm, and which is coated with multiple specific binding reactants such that the affinity constant of the nanoparticle towards an analyte exceeds that of free binding reactant towards the analyte and/or the association rate constant between the nanoparticle and the analyte exceeds the association rate constant between the free binding reactant and the analyte. Also disclosed is a homogenous assay based on a first group labeled with a luminescent energy donor nanoparticle and a second group labeled with an energy acceptor compound, where the donor has a long excited state lifetime, and the increase or decrease, respectively, in the energy transfer from the donor to the acceptor resulting from shortening or lengthening, respectively, of the distance between these groups, is measured.

### FIELD OF THE INVENTION

The present invention relates to improvements in biochemical assays utilizing biospecific binding reactant-coated nanoparticles. The present invention also relates to improvements in proximity based homogeneous assays, which use time resolved detection of luminescence. The specific improvements relate to the adaptation of the high specific activity, long lifetime luminescent nanoparticles long as energy donors, utilization of the enhanced kinetical properties of the nanoparticles coated with biospecific binding reactant and the energy acceptors with exceptional spectral characteristics.

### BACKGROUND OF THE INVENTION

A number of assays based on bioaffinity or enzymatically catalyzed reactions have been developed to analyze biologically important compounds from various biological samples (such as serum, blood, plasma, saliva, urine, feces, seminal plasma, sweat, liquor, amniotic fluid, tissue homogenate, ascites, etc.), samples in environmental studies (waste water, soil samples), industrial processes (process solutions, products) and compound libraries (screening libraries which may comprise organic compounds, inorganic compounds, natural products, extracts of biological sources, biological proteins, peptides, or nucleotides, etc.). Some of these assays rely on specific bioaffinity recognition reactions, where generally natural biological binding components are used to form the specific binding assay (with biological binding components such as antibodies, natural hormone binding proteins, lectins, enzymes, receptors, DNA, RNA) or artificially produced binding compounds like genetically or chemically engineered antibodies, molded plastic imprint (molecular imprinting), LNA (locked nucleic acid) and PNA (peptide nucleic acid) etc. Such assays generally rely on a label to quantitate the formed complexes after recognition, a binding reaction and suitable separation (separations like precipitation and centrifugation, filtration, affinity collection to e.g. plastic surfaces such as coated assay tubes, slides or microparticles, solvent extraction, gel filtration, or other chromatographic systems, and so on). The quantitation of the label in a free or bound fraction enables the calculation of the analyte in the sample directly or indirectly, generally through use of a set of standards to which unknown samples are compared.

The principles of immunoassays have been thoroughly reviewed by Price and Newman (Price C P and Newman D J, Principles and Practice of Immunoassay, 1997, Macmillan Reference Ltd., London, UK). Strategies to improve the sensitivity of biochemical assays have included strong binding affinity, low non-specific binding of the labeled reactant and high specific-activity of the label. Binding affinities are limited e.g. in the case of antibodies by the immune response although antibody engineering and recombinant antibodies have been successfully employed to improve the affinity (Lamminmäki U et al. J. Mol. Biol. 1999, 291, 589-602; Eriksson S et al. Clin. Chem. 2000, 46, 658-66). Non-specific binding is commonly minimized using solid-phase blocking and bulk proteins in the assay buffer. Research efforts have also been directed to improve the specific-activity of the label using new label molecules and background noise reduction (Kricka U. Pure Appl. Chem. 1996, 68, 1825-30; Kricka U. Clin. Chem. 1999, 45, 453-8). However, only limited improvements in sensitivity have been introduced to conventional assays although amplifying labels (Evangelista R A et al. Anal. Biochem. 1991, 197, 214-24), multiple labeling (Morton R C and Diamandis E P. Anal. Chem. 1990, 62, 1841-5) or enhanced specific-activity (Xu Y Y et al. Analyst 1992, 117, 1061-9), have been applied.

Extensive theoretical studies have supported the development of an ambient analyte immunoassay in a two-step heterogeneous microspot immunoassay with superior sensitivity, if labels with a very high specific-activity are available (Ekins R P. Clin. Biochem. Revs. 1987, 8, 12-23). Obviously, the development of supersensitive immunoassays requires, in addition to the methodological advances, improvements in the ordinary limiting factors, including strong binding affinity, low non-specific binding and high specific-activity. Homogeneous, luminescent oxygen channeling immunoassays (Ullman E F et al. Clin. Chem. 1996, 42, 1518-26) (LOCI™), heterogeneous, multianalyte microspot immunoassays (Ekins R P and Chu F W. Clin. Chem. 1991, 37, 1955-67; Ekins R P and Chu F W. PCT Int. Appl. 1993, WO 9308472 A 1) (Microspot®) and particulate fluorescent-label immunoassays utilize nanoparticle-antibody bioconjugates as a labeled component. It has been stated, that the surface density of binding sites on the particulate developing conjugate is likely to represent an important determinant of the sensitivity in the microspot immunoassay (Ekins R P and Chu F W PCT Int. Appl. 1993, WO 9308472 A 1). The potential increase in the effective affinity was speculated to originate from multivalent binding of the developing binding material to an individual antigen molecule via two or more separate antibodies directed to different epitopes of a single antigen, although it was not stated whether that was applicable to given examples. On the other hand, Ullman E F et al. (Clin. Chem. 1996, 42, 1518-26) has shown that the association rate between two nanoparticles from which one is coated with digoxins and the other with anti-digoxin antibodies, increases in the LOCI® system. However, the interaction of the nanoparticles was a result of multiple binding of digoxin and anti-digoxin and not a result of a single-valent binding event (an interaction of one digoxin to one anti-digoxin antibody). In the particulate fluorescent-label immunoassay the multiple binding of the anti-mouse antibody coated nanoparticle tracer to many surface bound mouse antibody analytes has shown to increase the avidity of this assay set-up (Hall M et al. Anal. Biochem 1999, 272, 165-70.).

The history of colloidal nanoparticles as labels in solid-phase immunoassays originates from the development of sol-particle immunoassays (Leuvering J H W et al. J. Immunoassay 1980, 1, 77-91) and subsequent adaptation of disperse dye (Gribnau T C J et al. J. Chromatography 1986, 376, 175-89) and fluorescent nanoparticles (Saunders G C et al. Clin. Chem. 1985, 31, 2020-3). Nanoparticle based solid-phase assays have demonstrated sensitivity enhancements over conventional enzyme and radiolabels, contributing to detailed studies of the function of the nanoparticle-antibody bioconjugates in existing assay systems (Saunders G C et al. Clin. Chem. 1985, 31, 2020-3; Okano K et al. Anal. Biochem. 1992, 202, 120-5; Kubitschko S et al. Anal. Biochem. 1997, 253, 112-22; Hall M et al. Anal. Biochem. 1999, 272, 165-70) and development of new methodologies and labels (Frank D et al. U.S. patent 1981, U.S. Pat. No. 4,283,382; Chan W C W and Nie S. Science 1998, 281, 2016-8; Beverloo H B et al. Anal. Biochem. 1992, 203, 326-34; Ullman E F et al. Proc. Natl. Acad. Sci. USA 1994, 91, 5426-30; Schultz S et al. Proc. Natl. Acad. Sci. USA 2000, 97, 996-1001; Roberts D et al. J. Lumin. 1998, 79, 225-31; Zijlmans H J M A A et al. Anal. Biochem. 1999, 267, 30-6). Reactivity of the nanoparticle labels can be enhanced by higher antibody loading on the nanoparticle surface as demonstrated by Okano K et al. (Anal. Biochem. 1992, 202, 120-5). However, non-specific binding was increased with high antibody-density particles. The observed, enhanced binding affinity could readily be interpreted as multivalent binding of the large bioconjugates to the surface-bound analytes due to the long incubation time which leads to the dissociation of the analyte from the surface to the solution and hence after rebinding to the surface increases multivalent binding of the nanoparticle. Also the large size of the nanoparticle, 760 nm, apparently leads to multivalent binding.

Affinity enhancement of complexes with multiple valences compared to the original antibodies have been shown using various Fv fragment-IgG (Ito W et al. J. Biol. Chem. 1993, 268, 20668-75) and tetravalent Fv fragment-core streptavidin complexes (Kipriyanov S et al. Prot. Eng. 1996, 9, 203-11). At least a part of the increased affinity was due to an increased association rate constant, 3.5 fold higher for tetravalent scFv:streptavidin complex compared to monovalent Fv. A similar phenomenon has been described earlier for the ferritin protein with 24 identical subunits: single-valent binding affinity of the protein was 1.6 ·10¹⁰ M⁻¹, while the intrinsic affinity of an individual subunit was 6.7 ·10⁸ M⁻¹ (Hogg, P et al. J. Arch. Biochem. Biophys. 1987, 254, 92-101).

Avidin (streptavidin) conjugates have long been used in various immuno- and nucleic acid assays (Wilchek M and Bayer E A, editors. In Methods in Enzymol, 1990, 184). A number of different fluorophores and enzymes have been conjugated to avidin, which then reacts with a biotinylated biospecific binding reactant (Papanastasiou-Diamandi A et al. Clin. Chem. 1992, 38, 545-8). The extremely high affinity (˜10¹⁵) and specificity of biotin towards avidin has made possible the use of this platform (Green N M. In: Wilchek M and Bayer E A, editors. Methods in Enzymology 1990, 184, 51-67). In a number of analysis the use of biotin-avidin complex has led to a good assay performance when avidin has been labeled with enzymes or prompt fluorophores. To further improve the assay performance avidin has been coupled to larger molecules in order to increase the number of enzymes or fluorophores per a single binding event. Diamandis et al. have conjugated streptavidin to thyroglobulin, which was labeled previously with time-resolved fluorescent Eu-chelates (Diamandis E P. Clin Chem 1991, 37, 1486-91). The formed complex tracing the analyte is considered to be complicated and difficult to control because multiple binding of proteins, lanthanide ions and chelates are required to form the successful complex. Hall et al. and Vener et al. have conjugated streptavidin to a large tracer nanoparticle containing prompt fluorophores (Hall M et al. Anal. Biochem. 1999, 272, 165-70; Vener T I et al. Anal. Biochem. 1991, 198, 308-11). Vener et al. used large particles, 1.8 μm in diameter, to assay biotinylated target DNA on membranes in a petri dish improving the detection sensitivity of the assay (one hour incubation) more than three orders of magnitude compared to the assay where the tracer molecule was soluble pyronine G-labeled streptavidin. Hall et al. used two approaches to assay mouse antibodies. The biotinylated anti-mouse antibody was preincubated with 220-μm streptavidin nanoparticles. This complex was allowed to react with microtiter well surface-bound analyte for 20 hours. If the streptavidin nanoparticle was allowed to react with the microtiter-plate surface-bound complex: surface-capture antibody I analyte I biotinylated anti-mouse antibody, Hall et al. failed to demonstrate the feasibility of such an assay.

In a more conventional assay format, after the analyte incubation step, a washing step is introduced prior to the adding of the label molecule such as labeled streptavidin. The washing step is crucial in this assay format in which a biotinylated biospecific binding reactant such as a biotinylated antibody is used because the free biotinylated biospecific binding reactants bind to labeled streptavidin in solution. This would vary significantly the amount of free label molecule in solution causing a major error source in the assay In microtiter well type assay systems Vener et al. and Hall et al. used in their study with streptavidin nanoparticles a washing step prior to adding of the streptavidin-coated tracer particles (Hall M et al. Anal. Biochem. 1999, 272, 165-70; Vener T I et al. Anal. Biochem. 1991, 198, 308-11). Ullman et al. have used streptavidin nanoparticles in an assay without subjecting the nanoparticles to washing but this was realized in the homogenous LOCI® assay format where no washing steps are required contrary to heterogeneous assays (Ullman E F et al. Clin. Chem. 1996, 42, 1518-26).

In a dissociation enhanced lanthanide fluoroimmunoassay (DELFIA®) lanthanide ions are dissociated from the chelate used for labeling of the tracer molecules. The lanthanide ions form in the solution a new fluorescent complex (Hemmilä I et al. Anal Biochem. 1984; 137:335-43). Alternative methods are described in literature where the lanthanide ions are not released from the chelate (Mukkala V-M et al. Helvetica Chim. Acta 1993, 76, 1361-78; Härmä H et al. Anal. Chim. Acta 2000, 410, 85-96). In these assay formats the analyte-bound intrinsically fluorescent chelate-labeled antibody is detected directly on the surface after a wash step.

Although sensitive assays can be run using these label techniques they still suffer from low signal levels. In addition, the intrinsically fluorescent chelates and generally all fluorophores are extremely sensitive to environmental changes. A means of decreasing the environmental effects is to have strict control over measurement conditions. In the All-In-One immunoassay concept controlling is made possible by drying the microtiter wells prior to detection (Lövgren T et al. Clin. Chem. 1996, 42, 1196-201). Water is known to quench luminescence and hence drying increases the signal level and reduces detection variations.

Colloidal stability of nanosized particles is of outmost importance to ensure non-aggregated particle suspensions (Griffin C et al. Microparticle Reagent Optimization, A laboratory reference manual. Seradyn, Particle Technology. Indianapolis, Ind.). Latex particles are known to flocculate easily due to hydrophobic interaction in-between particles and lacking of repulsive forces. Surface groups have been introduced on the particles to decrease a tendency to flocculate. One of the most effective means to increase repulsive forces is the introduction of carboxyl acid groups on the surface. These groups effectively repel one another when deprotonated in a moderate pH range. In an agglutination test the number of these functional groups may not be high due to the fact that the desired agglutination of the particle would not occur readily. However, when an agglutination test is not of interest and the aim is to have a nanosized particle react with a solid-phase surface-bound analyte, a higher repulsive force is preferred. This can be accomplished for example by introducing many functional groups on the nanoparticle and, hence, reducing apparent agglutination and also nonspecific binding to the solid-phase.

Proximity based homogeneous assays, which use time resolved detection of luminescence known to prior art are e.g. fluorescence polarization assays applied for small molecular compounds, enzyme-monitored immunoassays (Syva Co.), various fluorescence quenching or enhancing assays (for a review see e.g. Hernmilä, Applications of Fluorescence in Immunoassays, Wiley, NY, 1991). Other means to produce signal directly include the scintillation proximity principle (Amersham Pharmacia Biotech), which is based on short distance penetration of radiation particles in assay medium and a solid scintillator coated with catching reagents (Anal Biochem, (1987) 161, 494) and ALPHAscreen (BioSignal Packard) technology based on photosensitized formation of singlet oxygen, which migrates from a nanoparticle containing photosensitizer to an another nanoparticle containing chemiluminescer and generates delayed luminescence emission (Clin. Chem, (1996) 42, 1518). Another category of simplified assay technologies is the nonseparation assays, which, similarly to homogenous assays, avoid separation and washing steps. A true example of this kind of technology is microvolume assay technology based on two photon excitation and microparticle solid phase (Nat. Biotechnol., (2000), 18, 548). Also other similar nonseparation assay technologies exists (for a review see e.g. Mesa, Drug Disovery Today, 2000, 1:38-41).

Regardless of a great number of homogeneous assay designs published to day (for a review, see Ullmann, 1999, J. Chem. Ed. 76:781-788), there are no assays, where the versatility and sensitivity would match those of a good separation assay. The reason to that is manifold relating to e.g. the different way a homogeneous, versus heterogeneous, assay has to be optimized, the control of low affinity nonspecific bindings, and the limitations of applicability of most of the existing homogenous assay techniques. In addition, the conventional homogeneous fluorometric assays are very vulnerable to background interferences derived from various components in the samples. Fluorescence polarizations assays are interfered by low affinity nonspecific bindings (e.g. probe binding to albumin) and autofluorescence of samples.

Time-resolved (TR) fluorometry (time resolution in time-domain at micro- or millisecond range) is a perfect measuring regime for homogeneous assays, because it can totally discriminate the background fluorescence derived from organic compounds. When long enough delay times (time between pulsed excitation and starting of emission recording) can be used, all background interferences can be eliminated (for a review see. e.g. Hemmilä (1991); Gudgin Dickinson et al, (1995) J Photochem Photobiol 27, 3). In addition to separation based assays, also a number of homogeneous time resolved fluorometric assays have been described and patented (Mathis (1995) Clin Chem, 41, 1391; Selvin et al. (1994) Proc Natl Acad Sci, USA, 91, 10024, Hemmilä et. al (1996, 1999) WO 98/15830 and EP 0973 036 A2) with their limitations and drawbacks.

The complex compounds (chelates) developed relate to various types of multidentate complexes, i.e. chelates. According to various researches they have got different names, but all are based on organometallic complexes derived from a chelated lanthanide ion and a multidentate ligand. The names include supramolecular compounds, complexes, chelates, complexones, cryptates, crown-ether complexes, calixarenes, mixed-ligand complexes and so on.

There are a great number of stable fluorescent chelates, described in patents and articles, which could be used in time-resolved FRET assays, for example those mentioned in the following U.S. Pat. Nos. 4,761,481; 5,032,677; 5,055,578; 5,106,957; 5,116,989; 4,761,481; 4,801,722; 4,794,191; 4,637,988; 4,670,572; 4,837,169 and 4,859,777. The preferred chelate is composed of a nona-dentate chelating ligand, such as terpyridine (EP-A 403593; U.S. Pat. No. 5,324,825; U.S. Pat. No. 5,202,423; U.S. Pat. No. 5,316,909) or a terpyridine analogue with one or two five-membered rings (e.g. pyrazole, thiazole, triazine) (EP 077061041 and WO 93/11433). Very well suited chelates are also mentioned in the following articles: Takalo et al (1994) Bioconjugate Chem, 5, 278; Mukkala et at (1993) Helv Chim Acta, 76, 1361; Remuinnan et al (1993) J Chem Soc Perkin Trans, 2, 1099; Mukkala et al (1996) Helv Chim Acta, 79, 295; Takalo et al (1996) Helv Chim Acta, 79.

In addition fluorescent latex particles, containing fluorescent chelates, have been described as labels (Frank and Sundberg, 1978, U.S. Pat. No. 4,283,382, 1979, U.S. Pat. No. 425,313; Schaeffer et. al., 1985, U.S. Pat. No. 4,735,907, 1987, U.S. Pat. No. 4,784,912, Burdick and Danielson, 1989, U.S. Pat. No. 4,801,504, also method to prepare as Sutton et al., 1992, U.S. Pat. No. 5,234,841). The polymer inside particle stabilizes fluorescent chelates and prevents environmental effect to lanthanide fluorescence. This method also enables the use of unconjugateable or otherwise unsuitable chelates as labels. Fluorescent latex can be very densely packed with lanthanide chelates as they do not have any self quenching in high concentrations. The selection of chelates with best possible luminescent properties enables also superior fluorescent properties. No applications of fluorescent latex particles in FRET assays exists, since the long lifetime fluorescent background at the emission wavelength of the acceptor also increases relatively and apparently no advantage can be achieved. The same problem applies also to liposome labels containing fluorescent europium chelate (for example of europium liposome as donor and allophycocyanin as acceptor, see Okabayahi and Ikeuchi, 1998, Analyst 123:1329-1332).

Particulate fluorescent compounds with large and controllable Stoke's shift, very suitable to resonance energy transfer acceptor, have been introduced. Intramolecular energy transfer in particles using multiple fluorescent compounds embedded in polymeric matrix enables production of novel labels with desired spectral properties (see Buechler et al, 1998, U.S. Pat. No. 5,763,189; Singer and Haugland, 1996, U.S. Pat. No. 5,573,909; Roberts et al, 1998, J. Luminescence 79:225-231). Normal infrared chromophores have usually low solubility but embedding in polymeric matrix with soluble surface will enable also their use. Another class of particulate fluorescent compounds, semiconductor nanocrystals (see e.g. Bruchez et. al., 1998, Science 281:2013-2015), have size tunable emission wavelength and are excited efficiently at any wavelength shorter than the emission peak. These nanocrystals, also known as quantum dots, have same characteristic narrow, symmetric emission spectrum regardless of the excitation wavelength and emission wavelengths can be tuned from visible up to infrared (see e.g. Bailey, Chan and Nie, 2000, Near-Infrared-Emitting nanocrystals as biological labels, Abstract, Pittcon 2000 Symposium: Emerging Nanotechnologies for Chemical Analysis). Near-infrared emission is especially advantageous for analytical applications due to relatively low background and low absorbance in biological matrix (see e.g. Patonay et al., 2000, Near infrared absorption and fluorescence spectroscopy in analytical chemistry: moving to longer wavelengths, Abstract, Pittcon 2000). Quantum dots have been used as efficient donors because they are highly luminescent (1 quantum dot=20 organic dye molecules) and can be excited at any wavelength shorted than the emission peak (see Jain et al, 2000, Semiconductor Quantum dots for ultrasensitive FRET, Abstract, Pittcon 2000). In principle this phenomenon causes serious problems if quantum dots are used as acceptors in resonance energy transfer without temporal resolution.

The principle of a time-resolved homogeneous assay based on a specific energy transfer between a long lifetime donor and a short lifetime emitting acceptor molecule is summarized in FIG. 1. In the complex, when donor and acceptor labels are in proximity, the donor energy (D) excited by a short light pulse (A) is transferred by resonance energy transfer to acceptor. The energy transfer excited acceptor emission (AE) can be distinguished from the acceptor emission (B) excited directly by the light pulse (A) by applying a delay time (d) during which the counts from the photomultiplier tube are not recorded. Delayed emission from the donor (D) has a different wavelength than the sensitized (energy transfer excited) emission of the acceptor (AE), which enables a combination of spectral and temporal separation of signals. Hence, in homogenous bioaffinity assays (receptor-ligand binding, hybridization reaction, immunobinding, enzyme substrate binding etc.) the association or dissociation of donor-acceptor pairs can be followed by measuring the increase or decrease, respectively, in the signal from the energy transfer excited acceptor.

### OBJECTS AND SUMMARY OF THE INVENTION

An object of the present invention is to provide a nanoparticle useful for an assay to determine an analyte.

Another object of the present invention is to provide an improved assay for determining an analyte using said nanoparticle.

Yet another object of the present invention is to provide an improved proximity based homogenous assay.

Thus the present invention provides a nanoparticle comprising a specific binding reactant, said nanoparticle being useful for determining an analyte to which analyte or complex comprising said analyte said binding reactant is specific. The nanoparticle has the following characteristics:

a) the diameter of said nanoparticle is less than 200 nm, preferably less than 120 nm,
b) said nanoparticle is coated with multiple said specific binding reactants to the extent that
i) the affinity constant of said nanoparticle towards said analyte essentially exceeds that of free said binding reactant towards said analyte, and/or
ii) the association rate constant between said nanoparticle and said analyte essentially exceeds the association rate constant between free said binding reactant and said analyte; and
c) said nanoparticle comprises a detectable feature.

The present invention further provides an assay for determining an analyte to which analyte or complex comprising said analyte a binding reactant is specific wherein said assay utilizes a nanoparticle comprising said specific binding reactant. The nanoparticle utilized has the following characteristics:

a) the diameter of said nanoparticle is less than 200 nm, preferably less than 120 nm,
b) said nanoparticle is coated with multiple said specific binding reactants to the extent that
i) the affinity constant of said nanoparticle towards said analyte essentially exceeds that of free said binding reactant towards said analyte, and/or
ii) the association rate constant between said nanoparticle and said analyte essentially exceeds the association rate constant between free said binding reactant and said analyte; and
   c) said nanoparticle comprises a detectable feature.

The present invention also provides a proximity based homogenous assay comprising a first group labeled with an energy donating compound (donor) and a second group labeled with an energy accepting compound (acceptor), wherein
the donor is luminescent and has a long excited state lifetime and the acceptor is luminescent having a short or long excited state lifetime or the acceptor is non-luminescent, and
the increase or decrease, respectively, in the energy transfer from the donor to the acceptor resulting from shortening or lengthening, respectively, of the distance between said groups, is measured.

Characteristic for the assay is that the donor is a nanoparticle.

### BRIEF DESCRIPTION OF THE DRAWINGS

FIG. 1 shows the principle of a time-resolved homogeneous assay.

FIG. 2 shows a simulation of an assay demonstrating association, dissociation and complex concentration relevant to the assay as a function of reaction time.

FIG. 3 shows kinetic curves of a prostate-specific antigen (PSA) assay using varying numbers of nanoparticles.

FIG. 4 shows calibration curves of a PSA assay with and without a wash step.

FIG. 5 shows calibration curves for a PSA assay using varying numbers of nanoparticles.

FIG. 6 shows background fluorescence of a PSA assay using varying numbers of nanoparticles.

FIG. 7 shows determination of association rate constants of a PSA assay without nanoparticles and using nanoparticles with varying numbers of active binding sites per nanoparticle.

FIG. 8 shows dissociation kinetics for bioconjugates without nanoparticles and with nanoparticles with varying numbers of binding sites.

FIG. 9 shows determination of affinity of bioconjugates without nanoparticles and with nanoparticles with varying numbers of binding sites.

FIG. 10 shows standard curves for bioconjugate and labeled antibody based two-step, non-competitive immunoassays of free PSA.

FIG. 11 shows the effect of using two biotinylated antibodies instead of one on the kinetic curves of a PSA assay.

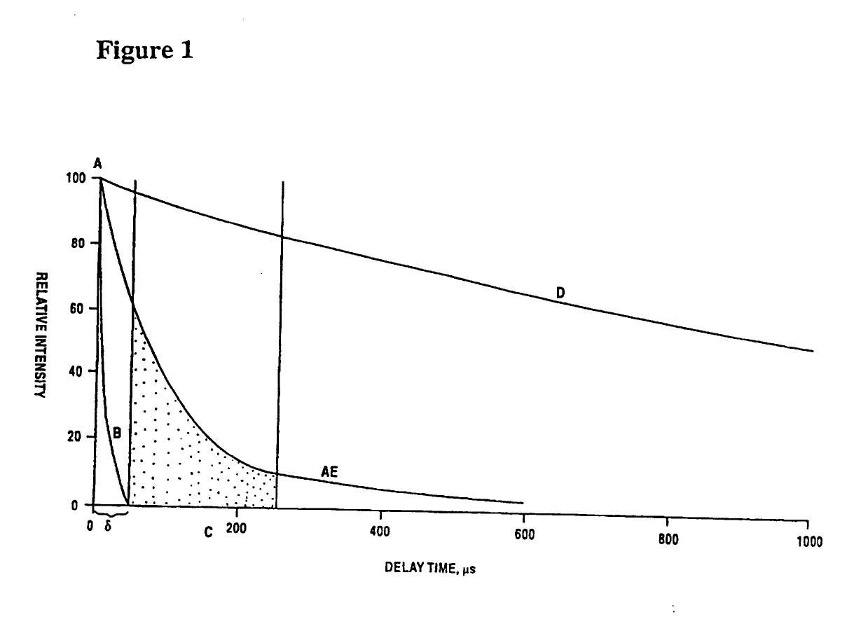

### DETAILED DESCRIPTION OF THE PREFERRED EMBODIMENTS

In the context of this application the term “nanoparticle” refers to any particle the average diameter of which is in the nanometer range, i.e. having an average diameter up to 1 μm.

In the context of this application the term specific binding reactant refers to any reactant that can be considered to be specific to any compound of relevance in the circumstances referred to. Specific binding reactants are e.g. an antibody, an antigen, a receptor ligand, a specific binding protein, protein A, protein G, avidin, avidin derivative, streptavidin, biotin, a nucleic acid, such as DNA, RNA, LNA (locked nucleic acid) and PNA (peptide nucleic acid), a peptide, a sugar, a hapten a virus a bacteria and a cell.

In the context of this application the term “detectable feature” refers to any feature making the entity comprising said “detectable feature” directly or indirectly qualitatively or quantitatively detectable by any known means. A detectable feature is thus e.g. a label such as a luminescent label.

The expression “heterogenous assay” relates to an assay in which a separation or a washing step is required. The expression “homogenous assay” relates to an assay in which a separation or a washing step is not required.

The terms “first group” and “second group” shall be understood to include any component such as a bioaffinity recognition component (in reactions where the distance between the groups decreases, e.g. in bioaffinity reactions) or a part of a molecule or substrate (e.g. the distal ends of a peptide molecule the cleavage of which will separate the two labeled groups from each other).

The term “donor” is defined as a particulate (diameter 400 nm or below, preferential below 50 nm) luminescent compound with long lifetime emission at visible or infrared wavelengths. The donor can be a lanthanide luminescent nanoparticle, e.g. inorganic phosphor, having a long excited state lifetime or polymeric nanoparticle embedded with an energy donating lanthanide luminescent compound, e.g. lanthanide chelate, having a long excited state lifetime. This includes also lanthanide phosphors and upconverting phosphors.

The term “lanthanide” is defined as luminescent lanthanide ion with luminescence emission in visible or near-infrared or infrared wavelengths and long fluorescence decay, e.g. europium(III), terbium(III), samarium(III), dysprosium(III), ytterbium(III), erbium(III) and neodynium(III). Also platinum(III) and palladium(III) should be noted have similar spectral and temporal properties when complexed to phorphyrins.

The term “chelate” is defined as a coordination complex where the central ion is coordinated with at least two coordination bonds to a single ligand (multidentate ligand). These may be named by different principles, and names like chelates, supramolecular compounds, complexes, complexones etc. are used. Special types of chelates include macrocyclic complexes, crown ethers, cryptates, calixarenes, phorphyrins and so on.

The preferred size of the nanoparticle ranges from 1 to 200 nm in diameter. The nanoparticle used can be made of organic or inorganic matter such as any polymer, gold, silver, carbon, silica, CdSe or CdS.

The nanoparticle can emit light originating from excitation of the nanoparticle or scattering or through electric pulse or chemical reaction. The affinity of the biospecific binding reactants on the nanoparticle, which is used in specific bioaffinity assays such as immunoassays, hybridization assays, receptor-binding assays and cellular binding assays, e.g. utilizing luminescence (fluorescence, time-resolved fluorescence, phosphorescence, chemi-luminescence, bioluminescence) detection of the specific analyte, exceeds the affinity of said labeled soluble single biospecific binding reactant. Nanoparticles may or may not carry one or more luminescent molecules or molecules leading to luminescent emission inside the nanoparticle or on the surface of the nanoparticle using one or more of the following luminescent molecules or molecules leading to luminescent emission:
Time-resolved fluorescent labels e.g.
   Eu(III), Tb(III), Sm(III), Dy(III) chelates, i.e. lanthanide chelates,
   Pt and Pd porphyrin labels,
   lanthanide phosphors;
Upconverting fluorescent labels e.g.
   (Y.Yb.Er)O₂S
   (Y.Yb.Tm)₂O₂S
Rapidly decaying fluorescent labels e.g.
   fluorescein and fluorescein derivatives, rhodamine and rhodamine derivatives,
   CdS and CdSe nanocrystals
   green fluorescent protein and green fluorescent protein derivatives;
chemiluminescent labels e.g.
   dioxetane derivatives, alkaline phosphatase, β-galactosidase; and
bioluminescent labels
   alkaline phosphatase, β-galactosidase.

The biospecific binding reactant is attached to the nanoparticle through one or more of the following means: adsorption, covalent coupling, grafting, solid phase synthesis or another biospecific binding reactant. The preferred method is adsorption and covalent coupling.

The nanoparticle optionally contains one or more of functional groups on the surface. Such functional groups may include but not be limited to carboxyl (COOH), amino (NH2, NHR, NR1R2, NR1R1), aldehyde or ketone (CHO, CO), hydroxyl (OH) or thiol (SH).

The present invention enables performing biospecific assays with a biospecific binding reactant whose affinity exceeds the affinity of the same single, soluble biospecific binding reactant by introducing a number of biospecific binding reactants onto a nanoparticle. As the affinity of the biospecific binding reactant is increased kinetics and sensitivity of said biospecific assays are significantly improved compared to the same assay using a soluble labeled biospecific binding reactant. The nanoparticle coated with biospecific binding reactants can be used in heterogeneous as well as in homogenous assay formats. These assays can be either non-competitive or competitive. Assays utilizing nanoparticles can be used for simultaneous measurement of two or more analytes detected by a specific nanoparticle towards each analyte.

A heterogenous assay according to the invention can comprise the steps of

a) contacting a first binding reactant bound to a solid phase, which reactant is specific to a first binding site of said analyte, with a sample comprising said analyte;
b) optionally reacting said analyte with said first binding reactant;
c) adding to the composition obtained in step a) said nanoparticles comprising a second binding reactant, which reactant is specific to a second binding site of said analyte;
d) reacting second binding reactant of said nanoparticles with said analyte bound to first binding reactant bound to said solid phase;
e) washing said solid phase, which solid phase binds a first binding reactant bound to said analyte bound to second binding reactant of nanoparticles, essentially free of nanoparticles not biospecifically bound to said solid phase; and
f) detecting said nanoparticles bound to said solid phase to enable determination of said analyte.

If the optional reacting step b) is not carried out steps a) and c) are carried out essentially simultaneously.

Another heterogeneous assay according to the invention can comprise the steps of

a) contacting a first binding reactant bound to a solid phase, which reactant is specific to a first binding site of said analyte, with a sample comprising said analyte;
b) adding to the composition obtained in step a) a second binding reactant bound to a third binding reactant, which second binding reactant is specific to a second binding site of said analyte;
c) adding to the composition obtained in step b) said nanoparticles comprising a fourth binding reactant, which reactant is specific to said third binding reactant;
d) reacting said fourth binding reactant of said nanoparticles with third binding reactant bound to second binding reactant bound to said analyte bound to said first binding reactant bound to said solid phase;
e) washing said solid phase, which solid phase binds first binding reactant bound to analyte bound to second binding reactant bound to third binding reactant bound to fourth binding reactant of said nanoparticles, essentially free of nanoparticles not biospecifically bound to said solid phase; and
f) detecting said nanoparticles bound to said solid phase to enable determination of said analyte.

In this assay the second and third binding reactant can be the same entity, e.g. an antibody, having two different binding sites of which one is directed towards the analyte and the other towards the fourth binding reactant bound to the nanoparticle.

In this assay said third binding reactant is preferably biotin and said fourth binding reactant is preferably avidin or streptavidin. Alternatively preferred third binding reactant could be avidin or streptavidin and preferred fourth binding reactant biotin. It should also be noted that an assay comprising steps a) to f) above could also be applicable and efficient using any nanoparticle although the preferable choice would be a nanoparticle as referred to in step c) and defined by the claims of this application.

Yet another heterogeneous and competitive assay according to the invention could comprise the steps of

a) contacting a first binding reactant bound to a solid phase, which reactant is specific to a first binding site of said analyte, with a sample comprising said analyte, and with additional said analyte bound to said nanoparticles,
b) washing said solid phase, which solid phase binds first binding reactant bound to analyte bound to said nanoparticle, essentially free of nanoparticles not bound to said solid phase; and
c) detecting nanoparticles bound to said solid phase to enable determination of said analyte.

An alternative heterogeneous and competitive assay according to the invention could comprise the steps of

a) contacting a first binding reactant bound to a solid phase, which reactant is specific to a first binding site of said analyte, with a sample comprising said analyte, and with additional said analyte bound to a second binding reactant
b) adding to the composition obtained in step a) said nanoparticles comprising a third binding reactant, which reactant is specific to said second binding reactant,
c) reacting said third binding reactant of said nanoparticles with said second binding reactant bound to said additional analyte bound to first binding reactant bound to said solid phase;
d) washing said solid phase, which solid phase binds first binding reactant, bound to analyte bound to second binding reactant bound to third binding reactant of said nanoparticles, essentially free of nanoparticles not bound to said solid phase; and
e) detecting nanoparticles bound to said solid phase to enable determination of said analyte.

In a first non-competitive heterogeneous approach the analyte is added to a solid-phase. After a washing step, the nanoparticle coated with said biospecific binding reactants is incubated with the analyte bound on said solid-phase surface. After the final washing step, the luminescent signal is read directly from said solid-phase surface or after drying or after signal enhancement or after signal amplification.

In a second non-competitive heterogeneous approach the analyte is incubated together with the nanoparticle coated with said biospecific binding reactants in one-step onto said solid-phase surface-bound capture molecule. After the washing step, the luminescent signal is read directly from said solid-phase surface, after drying, after signal enhancement, or after signal amplification.

In a third non-competitive heterogeneous approach the analyte is incubated separately or together in one or two steps with a second analyte-specific binding reactant, optionally bound to a third binding reactant, onto said solid-phase surface-bound capture molecule.

After the washing step, the nanoparticle coated with said biospecific binding reactants is incubated with the analyte bound on said solid-phase surface. After another washing step, the nanoparticle coated with said biospecific binding reactant is incubated with the analyte bound on said solid-phase surface. After the final washing step, the luminescent signal is read directly from said solid-phase surface, after drying, after signal enhancement, or after signal amplification.

In a fourth non-competitive heterogeneous approach the analyte is incubated separately or together in one or two steps with said second analyte-specific binding reactant, optionally bound to a third binding reactant, onto said solid-phase surface-bound capture molecule. After analyte incubation, the washing step is omitted due to the number of said second analyte-specific binding molecules and the number of available said biospecific binding reactants on the nanoparticle in the reaction. In such an assay the number of said second analyte-specific binding molecules does not exceed the number of said biospecific binding reactant molecules on the surface of the nanoparticle coated with said biospecific binding reactant. In the excess of said nanoparticle-bound biospecific binding reactant the assay has proven not to be interfered by free said second analyte-specific binding molecule in solution although said non-analyte bound second analyte specific binding molecules may react with the nanoparticle coated with said biospecific binding reactant in solution prior to the reaction of the nanoparticle coated with said biospecific binding reactant. After the final washing step, the luminescent signal is read directly from said solid-phase surface, after drying, after signal enhancement, or after signal amplification.

In a fifth non-competitive heterogeneous approach the analyte is incubated separately or together in one or two steps with two or more said second analyte specific binding reactant, optionally bound to a third binding reactant, onto said solid-phase surface-bound capture molecule. Using more than one said second analyte specific binding molecule the dissociation of the said second analyte specific binding molecule or the nanoparticles coated with said biospecific binding reactant is reduced and a pseudo-equilibrium state is achieved. After analyte incubation, said solid-phase can be washed. After the final washing step, the luminescent signal is read directly from said solid-phase surface, after drying, after signal enhancement, or after signal amplification.

In a non-competitive assay utilizing said third assay approach, the dynamic range of said assay can be adjusted on the basis of the number of the nanoparticles coated with said biospecific binding reactants: the higher the number of the nanoparticles coated with said biospecific binding reactant the larger the dynamic range is, because the nonspecific binding of the assay is not increased when the number of nanoparticle coated with said biospecific binding reactants is increased.

In a preferred non-competitive heterogeneous assay concept utilizing the second or the third approach, the number of said second biospecific binding reactant molecules is lower than used to immobilize said biospecific binding reactant onto the nanoparticle in the first assay approach. The third, fourth and fifth assay concept significantly decreases the amount of the second biospecific binding reactant required in the assays.

In the second and third heterogeneous assay approach the incubation step of the nanoparticles coated with said biospecific binding reactants is carried out any time during non-equilibrium or equilibrium. In a typical second and third assay approach the incubation step of the nanoparticles coated with said biospecific binding reactant is carried out any time during non-equilibrium, more typically in less than two hours and preferable in less than one hour.

In a first competitive heterogeneous assay the analyte is added to said solid-phase together with a competing labeled analyte derivative or separately. The label is e.g. one of the following:
biotin, streptavidin, avidin or avidin derivative,
an antibody, protein A, protein G or an antigen.

After an optional washing step, the nanoparticles coated with said biospecific binding reactants are incubated with said labeled analyte bound on said solid-phase surface. After the final washing step, the luminescent signal is read directly from said solid-phase surface or after drying or after signal enhancement or after signal amplification.

In the second competitive heterogeneous assay format, the competing element in the analyte incubation step is the nanoparticle coated with an analyte or analyte-derivative molecules. In the assay format the number of the competing analyte or analyte-derivative molecules on the surface of the nanoparticle can be controlled which significantly improves the control over the assay.

In assays according to the invention exemplified above the non-optional reacting step is typically discontinued essentially before equilibrium. The duration of said non-optional reacting step is typically less than 2 h and preferably less than 1 h.

In assays according to the invention exemplified above the solid phase is typically a essentially flat surface of e.g. a microtiter well, the surface of a slide, the surface of a particle or the surface of a strip.

In assays according to the invention exemplified above the amount of nanoparticles added per each individual sample to be assayed is dependent on the assay volume and the size of the particle.

Heterogeneous assay according to the invention can thus include assays where, after incubation of the analyte and a second biospecific binding reactant such as a biotinylated antibody or anti-mouse antibody, the solid phase is not washed prior to adding the nanoparticle coated with said biospecific binding reactant.

Heterogeneous assay according to the invention also include assays where the analyte and a second biospecific binding reactant on the solid phase are traced with a nanoparticle coated with said biospecific binding reactant at any time during non-equilibrium and equilibrium state.

The invention also includes homogenous assays in which energy from a donor particle is transferred to one or more acceptor molecules or to one or more particles containing one or more acceptor molecules of the same or different types of acceptor molecules.

Preferred acceptor molecules are:
rapidly decaying fluorophores, e.g. APC, Cy5, Cy7, NN-382, FluoSpheres semiconducting materials, e.g. CdSe nanocrystals (i.e. Quantum Dots) fluorescent energy transfer complexes, e.g. TransFluoSpheres,
   Cy7-APC tandem dye, and
time-resolved fluorophores, e.g. ytterbium chelates, inorganic phosphors.

One or more of the same or different types of the said acceptor molecules may be attached to a biospecific binding reactant.

One or more type of the said acceptor molecules and one or more of the types of said acceptor molecules may be attached onto the surface of the nanoparticle coated with said biospecific binding reactant or embedded into the nanoparticle coated with said biospecific binding reactants.

The preferred size of the acceptor particle ranges from 1 nm to 1 mm in diameter.

In a preferred heterogeneous assay arrangement an improved assay performance is obtained using the mono-valent affinity of the nanoparticle coated with said biospecific binding reactants. In the assay the mono-valent affinity of the nanoparticle coated with said biospecific binding reactants exceed the mono-valent affinity of the soluble biospecific binding reactant. That is achieved by increasing the number of the binding sites of said biospecific binding reactants on the surface of the nanoparticle. This improvement in affinity has been proved to originate mainly from the increase in the rate of association and partially from the decrease in the rate of dissociation of biochemical analysis. The association rate has shown to increase nearly in a linear manner. In addition to the mono-valent analyte molecules, analytes with multiple binding sites, such as whole cells, bacteria, viruses and multimeric proteins, benefit from the use of nanoparticle label coated with said biospecific binding reactants because the affinity of nanoparticle coated with said biospecific binding reactants is higher towards multiple binding sites on the surface of the multi-binding site analytes. The improved affinity originates mainly from the reduced dissociation rate and partially from the improved association rate.

In the non-competitive or competitive assay format the analyte-specific capture molecule can be immobilized either directly onto the surface of a solid-phase or indirectly.

The assay system is fully functional whether single or aggregated nanoparticles are being used. Non-aggregated nanoparticles are preferred.

In a homogenous assay the analyte is incubated together with the donor nanoparticle coated with a first biospecific binding reactant and the acceptor molecule attached to a second biospecific binding reactant or a second biospecific binding reactant coated particle containing acceptor molecules. The luminescent signal is read directly from solution.

In a preferred homogenous assay arrangement an improved assay performance is obtained using the mono-valent affinity of the nanoparticle coated with said biospecific binding reactants. In the assay the mono-valent affinity of the nanoparticle coated with said biospecific binding reactants exceeds the mono-valent affinity of the soluble biospecific binding reactant. This is achieved by increasing the number of binding sites of said biospecific binding reactant on the surface of the nanoparticle.

The present invention also relates to improvement in proximity-based homogeneous assays, which use time-resolved detection of luminescence. The specific improvements relate to the increased specific activity of the nanoparticle donor, reduction of the long lifetime luminescent background at the emission wavelength of the acceptor using acceptor compounds with a large spectral separation of energy absorption (excitation) and luminescence emission, and utilization of the enhanced association rate constant and the affinity constant of the nanoparticle labeled biospecific binding reactant. The combination of high-specific activity of the long lifetime nanoparticle donor and large Stoke's shift of acceptor allows detection of lower number of complexes in assays where association or dissociation is to be followed, i.e. label pari distance shortening or lengthening, than has been possible with earlier described homogeneous methods using time-resolved detection of luminescence.

On the contrary to the way the donor has been chosen in all prior art methods, the donor used in the present invention is a resonance energy transfer donor, a light emitting lanthanide containing particulate compound with high specific activity wherein the acceptor is selected to have exceptionally wide Stokes's shift between energy absorption and energy emission to avoid practically all long lifetime fluorescent background from donor at the emission wavelength of the acceptor. The improved proximity-based homogeneous time-resolved luminescence assay comprises one group labeled with a energy donating luminescent nanoparticle (donor) having a long excited state lifetime or nanoparticle embedded with an energy donating luminescent compound (donor) having a long excited state lifetime and an another group labeled with an energy accepting luminescent compound (acceptor) having either a short or long excited state lifetime or with a non-luminescent compound.

Characteristic for the invention is that the improvements enable detection of the increase or decrease in the energy transfer from the donor to the acceptor resulting from shortening or lengthening, respectively, of the distance between said groups in response to presence of a minor quantity of assayed group or activity. The acceptor is typically luminescent and the luminescence of the acceptor is preferably measured at a wavelength were the donor has no luminescence or essentially no luminescence, i.e. the luminescence of the donor is not significant compared to background luminescence.

The lanthanides have several ground states giving rise to numerous transitions in their emissions. Regardless of the fact that emissions are sharp and well defined, there always tends to be a minor background at the wavelength acceptors are measured. The relative background is, however, less a problem at longer wavelengths. e.g. with Eu there are areas were Eu has a very minor background between 700 and 800 nm and at over 800 nm Eu does not emit any direct emission. With Tb the extended wavelength range gives the possibility to use acceptors emitting at over 700 nm, where Tb does not create any background. By choosing a non-overlapping wavelength area, the sensitivity and dynamic range of time resolved fluorescence energy transfer can be improved since the long life-time fluorescence background is low. The donor has to have high specific activity to produce detectable acceptor emission after energy transfer and using conventional time-resolved fluorophores, e.g. fluorescent chelates, improved sensitivity may not be achieved.

In assays where association is to be measured and a luminescent, short decay time acceptor and a long decay time donor are used, the emission of acceptor molecules is followed using a delay time i.e. time-resolved fluorometry to avoid the interference of the acceptors direct luminescence (emanating from direct excitation of acceptor). It is desirable to construct the assay in such a way that acceptor molecules are in excess (with time-resolved mode, their interference is negligible) and the energy tranfer between donor and acceptor creates an increase in signal.

The sensitivity of any energy transfer based assay depends on both the intensity level of the obtained signal and on the total background. The signal level in a particular assay depends on the used chelate, its total excited state population and duration in the complex. The excited state population is a direct function of luminescent properties of the chelate, i.e. molar absorptivity (e), quantum yield (f) and decay time (t). Accordingly a preferred donor has to have very high luminescence yield (significantly higher than particularly expressed in prior art, WO/98/15830) and long excited state lifetime (preferably over 1 ins).

A preferred acceptor molecule for association assays is highly luminescent (with quantum yield as near unity (1) as possible) with a high molar absorption coefficient (preferably over 100 000) at donor emission wavelength. It is important that the acceptor has a high quantum yield, and emits light at wavelength where the used lanthanide has a negligible background.

Preferably, the instrument automatically corrects any attenuation of excitation the sample may cause by simultaneously following the absorbance of the samples diluted into assay mixture and correcting the emission readings according to excitation or emission attenuation by sample absorption.

The present invention relates to improvements in assay performance using a nanoparticle coated with a biospecific binding reactant. The specific improvement relates to the increment in association rate and thus in affinity of said biospecific binding reactant when multiple said biospecific binding reactants are coated on the nanoparticle increasing the number of binding sites. In addition, the specific improvement relates to the means of performing said biospecific assays using the nanoparticle coated with said biospecific binding reactant.

Advantages of assays according to the invention are:

The affinity of said soluble specific binding reactant can be improved significantly by introducing a sufficient amount of said specific binding reactant onto a nanoparticle.

The association rate of the nanoparticle coated with said specific binding reactant reaches or exceeds that of said soluble specific binding reactant.

The dissociation rate of the nanoparticle coated with said specific binding reactant is lower than that of said soluble specific binding reactant.

The nanoparticle contains a high amount of luminescent label and has a very high specific activity.

The time-resolved fluorescent label inside the nanoparticle has no quenching effect even in very high concentration contrary to the rapidly decaying fluorophores.

The nanoparticle is highly insensitive to environmental effects caused by water, quenchers or oxygen.

The nanoparticle can be detected directly on a surface without elimination of said environmental effects.

The nanoparticle makes very sensitive biospecific assays possible.

The nanoparticle can be used to detect single molecules.

In an assay where an analyte and a second biospecific binding reactant react with a first solid-phase surface-hound capture biospecific binding reactant prior to the addition of the nanoparticles coated with a third biospecific binding reactant less of second biospecific binding reactant can be used in the assay reducing drastically the amount of the second biospecific binding reactant required in the nanoparticle based assay.

When an analyte and a first biospecific binding reactant react with a solid-phase surface bound capture biospecific binding reactant prior to the addition of nanoparticles coated with a second biospecific binding reactant a very rapid, less than 5 minutes, incubation step of the nanoparticle coated with second biospecific binding reactant can be used.

In an assay where an analyte and a first biospecific binding reactant react with a solid-phase surface bound capture biospecific binding reactant prior to the addition of nanoparticles coated with a second biospecific binding reactant the dynamic range of the assay can be adjusted on based on the amount of the nanoparticles coated with second biospecific binding reactant without affecting non-specific binding of the nanoparticles coated with second biospecific binding reactant to the solid-phase surface.

The nanoparticle is small in size and thus does not settle readily.

The nanoparticle can contain functional groups on the surface through which specific binding reactants can be coupled covalently on the nanoparticle.

The nanoparticle coated with said specific binding reactant is potentially a better solution for a label because a conventionally labeled (generally more than 5 labels per molecule) protein can significantly interfere with binding affinity and non-specific binding of the protein which is not the case with the nanoparticle based label where the large surface area ensures that a substantial number of binding sites are available for binding of the analyte. This effect is especially minimized using a third specific binding reactant such as a site-specifically biotinylated antibody fragment.

When a solid-phase surface is coated with an antibody without using any denaturating step or partially denaturating step prior to the coating procedure nonspecific binding of the nanoparticles coated with said biospecific binding reactant to the coated solid-phase surface is significantly reduced.

If smaller fragments of antibodies are coated on nanoparticles the number of binding sites can be increased and the affinity of the tracer molecule can be further improved.

The amount of biospecific binding reactants in an assays can be increased by coating more said biospecific binding reactant onto a nanoparticle whereas the number of label molecules remain the same which is contrary to typical prior art biospecific assays where labeled, soluble said biospecific binding reactants are used because by increasing the number of labeled, soluble said biospecific binding reactants the amount of label is increased accordingly.

The nanoparticle can be used as a donor molecule in a homogenous assay.

The donor nanoparticle typically yields a high background signal when used in an energy transfer process. This can be circumvented by transferring the energy far enough by using suitable acceptor molecules or particles containing acceptor molecules.

Due to the very broad absorption spectrum quantum dots are not suitable as acceptor molecules when rapidly decaying dyes are used since quantum dots are excited simultaneously with donor molecules and no specific energy transfer occurs whereas using donor dyes with a very long exited state lifetime specific energy transfer to acceptor quantum dots can be detected.

Nanoparticles as labels offer an advantage to control the size of the label molecule and hence homogenize the used label component contrary to conjugated molecules such as a streptavidin-thyroglobulin-based label.

When streptavidin-coated nanoparticles are used in the assay, a washing step is not required since the number of label molecules (nanoparticles) is not affected by the free biotinylated biospecific binding reactant. Although the free biotinylated biospecific binding reactant binds to the streptavidin-coated nanoparticle the high number of available streptavidin binding sites still enables to carry out of the assay without detrimental loss in assay performance.

Labeling of proteins through amino groups is generally a random process, which leads to an uncontrolled number of labels per protein. In approaches where the number of labels per molecules have been increased such as demonstrated by Diamandis et al., the same problem of randomness is encountered (Diamandis E P. Clin Chem 1991, 37, 1486-91). The degree of labeling is, however, more readily controlled when fluorophores are embedded into a particle.

The number of proteins on nanoparticles should be controlled to obtain a nanoparticle that behaves optimally as a tracer molecule in an assay. Simultaneous control of proteins and labels when a larger protein/label-complex is formed is very difficult. However, using a nanoparticle the number of proteins and the amount of labels can be controlled. Moreover, when surface-active groups are present such as COOH, controlling of the number of proteins can be done by controlling the activated sites on the surface of a nanoparticle. By activating only a limited number of surface groups more repulsive groups are left on the surface to increase the zeta potential of the particle and hence also nonspecific binding is decreased.

### Experimental Section

### Nanoparticle-Antibody and Nanoparticle-Streptavidin Bioconjugates

Monoclonal antibody, Mab5A10 or streptavidin was covalently coupled to activated nanoparticles mainly by primary amine groups using two-step EDAC (N-(3-dimethylaminopropyl)-N′-ethylcarbodiimide, Fluka, Buchs, Switzerland) and sulfo-NHS (N-hydroxysulfosuccinimide, Fluka, Buchs, Switzerland) coupling chemistry. Nanoparticles were pre-washed with 25 mmol l⁻¹ phosphate buffer, pH 7.0, using Amicon ultrafiltration stirred cell (Millipore, Bedford, Mass.) equipped with 500 kD polyethersulfone ultrafiltration membrane (Millipore) and resuspended in phosphate buffer using Labsonic U (B. Braun, Melsungen, Germany) tip sonicator (10 seconds, 80 W power level). Carboxyl groups were activated by incubating nanoparticles 15 min in phosphate buffer containing 2 mmol l⁻¹ EDAC and 100 mmol l⁻¹ sulfo-NHS. Activated nanoparticles were washed with 25 mmol l⁻¹ carbonate buffer, pH 8.5, and mixed to carbonate buffer containing Mab5A10 or streptavidin. Nanoparticle concentration in coupling reaction was 0.4 w/v %. Mab5A10 concentrations varied from 5 to 0.078 g l⁻¹ and the streptavidin concentration was 0.9 mg l⁻¹. Coupling reaction was incubated for 2 h with slow shaking, and thereafter 1% bovine serum albumin was added to block remaining active groups for 15 min. Nanoparticle-antibody bioconjugates were washed six times with 2 mmol l⁻¹ Tris-HCl, pH 8.0, containing 0.01% Tween 40 and 50 mmol l⁻¹ salicylic acid, and finally resuspensed to same buffer containing additionally 0.1% gelatin and 0.1% Tween 85. The suspension was centrifuged twice at 2500 g for 5 min to separate non-colloidal aggregates from monodisperse suspension and stored at +4° C.

### Binding Site Number

The number of active binding sites of covalently coupled Mab5A10 on a single nanoparticle-antibody bioconjugate was determined using Tb(III)-N1-ITC labeled PSA (Prostate-Specific Antigen) and measuring the ratio between terbium(m) fluorescence from particle-bound labeled PSA and europium(III) fluorescence from nanoparticles. Nanoparticles (6·10¹⁰ pcs ml⁻¹) were incubated for 1 h with slow shaking in the assay buffer (PerkinElmer Life Sciences, Wallac Oy, Turku, Finland) containing 3.3 mg Tb(III)-N1-ITC labeled PSA, 0.0005 w/v % milk powder and 0.005 w/v % Tween 85. Nanoparticles and particle bound labeled PSA were separated from unbound labeled PSA by size-exclusion chromatography using Sepharose 6B (Pharmacia Amersham, Uppsala, Sweden) matrix and 10 mmol l⁻¹ Tris-HCl buffer, pH 7.8, containing 0.9% NaCl and 0.01Tween 20. Nanoparticle-antibody bioconjugate fractions were diluted to DELFIA® enhancement solution and europium(III) fluorescence was compared to nanoparticle standard to calculate nanoparticle concentrations. Terbium(III) fluorescence from the same fractions and terbium(III) standard solution were measured after additional incubation with DELFIA® enhancer, and the number of active binding sites was calculated from the number of terbium(III) ions per nanoparticle divided by the labeling degree of PSA. Non-specific binding of the labeled PSA was controlled using non-coated nanoparticles blocked with bovine serum albumin. Both europium(III) and terbium(III) fluorescence were measured using a Victor™ 1420 fluorometer in time-resolved mode, at 613 nm with narrow emission aperture and at 545 nm, respectively.

The number of streptavidin molecules on a single nanoparticle was determined using site-specifically biotinylated Fab-5A10 fragment and Tb(III)-N1-ITC labeled PSA. Nanoparticles (33 pmol were incubated with 33 nmol l⁻¹ of biotinylated Fab-5A10 and 150 nmol l⁻¹ of Tb(III)-N1-ITC labeled PSA in 100 μl of assay buffer for 1 h at room temperature. Thereafter the nanoparticles were separated from unbound Tb(III)-N1-ITC labeled PSA and measured as indicated above for Mab5A10. Eventually, the number of streptavidin molecules was calculated assuming that one Tb-PSA reacted with one Fab-5A 10 fragment, which, in turn, corresponded to one streptavidin molecule.

### Biotinylation of Mab-5 A10

Mab-5A10 (400 mg l⁻¹) was biotinylated with 350 mmol l¹ of biotin-PEG-CO2-NHS (Shearwater Polymers, Huntsville, Ala.) in 50 mmol l⁻¹ carbonate buffer, pH 9.8, for 2 h at room temperature. The biotinylated Mab was purified from unbound biotin reagent with NAP-5 and NAP-10 columns (Pharmacia Amersham Biotech). The elution was carried out with 50 Tris buffer, pH 7, including 150 mmol I′ of NaCl.

### EXAMPLES

Table 1 shows luminescence transitions of Eu³⁺. Excited state ⁵D₁ takes part in energy transfer from ligand to ion, and ⁵D₀ is the major emittive level. Direct transitions from ⁵D¹ are short-lived and much weaker. The lanthanide ions have several ground states giving rise to numerous transitions in their emission. Regardless of the fact that the emissions are sharp and well defined, there always tends to be a minor relative background emission at the wavelength acceptor being measured. An Eu³⁺ ion has only very weak emission above 710 nm and no detectable luminescence emission above 820 nm. In the case of Tb³⁺ ion no luminescence emission above 700 nm exists.

Table 2 shows an example in which the increase of the number of binding sites of a nanoparticle-antibody bioconjugate increases the affinity constant as well as the association rate constant. In this example the affinity constant exceeds that of the labeled antibody when the number of binding sites increases from 12 to 19 whereas the association rate constant exceeds that of the labeled antibody when the number of binding sites increases from 46 to 76.

TABLE 1
Europium(III) luminescence transitions

| Transition | Emission wavelength (nm) | Relative intensity |
|---|---|---|
| ⁵D₁ -> ⁷F₀ | 526 | very weak |
| ⁵D₁ -> ⁷F₁ | 537 | very weak |
| ⁵D₁ -> ⁷F₂ | 558-560 | very weak |
| ⁵D₀ -> ⁷F₀ | 578-580 | weak |
| ⁵D₀ -> ⁷F₁ | 585-600 | strong |
| ⁵D₀ -> ⁷F₂ | 610-630 | strongest |
| ⁵D₀ -> ⁷F₃ | 645-660 | weak |
| ⁵D₀ -> ⁷F₄ | 680-705 | medium |
| ⁵D₀ -> ⁷F₅ | 751 | very weak |
| ⁵D₀ -> ⁷F₆ | 815 | very weak |

TABLE 2
Affinity and kinetic rate constants for the labeled compounds.

| | | | | |
|---|---|---|---|---|
| | mono-valent | association | dissociation | |
| labeled | affinity constant | rale constant | rate constant | affinity constant |
| compound | Kₐ (M⁻¹/1E9)^{a} | kₐ (M⁻¹ s⁻¹/1E5)^{a,c} | K_{d} (s⁻¹/1E−5)^{a} | Kₐ (M⁻¹/1E9)^{b} |

Eu(III)-N1-ITC labeled antibody

| | | | | |
|---|---|---|---|---|
| Mab5A10 | 6.6 ± 0.3 | 12.6 ± 0.7 | 17.6 ± 0.8 | 7.2 |

number of binding sites for the nanoparticle-antibody bioconjugates

| | | | | |
|---|---|---|---|---|
| 130 | 53.8 ± 7.9 | 24.8 ± 1.6 | 3.7 ± 0.9 | 67.8 |
| 76 | 35.1 ± 5.7 | 15.3 ± 0.9 | 3.9 ± 1.5 | 39.4 |
| 46 | 22.6 ± 3.0 | 11.8 ± 0.6 | 4.5 ± 1.4 | 25.9 |
| 30 | 13.3 ± 1.7 | 8.3 ± 0.3 | 6.0 ± 1.3 | 13.9 |
| 19 | 9.3 ± 0.7 | 6.1 ± 0.3 | 6.1 ± 1.4 | 10.0 |
| 12 | 4.1 ± 0.9 | 4.1 ± 0.1 | 5.3 ± 1.0 | 7.8 |
| 8 | 1.6 ± 0.5 | 2.8 ± 0.1 | 6.9 ± 0.9 | 4.0 |

^{a}Mean ± SD
^{b}Affinity constant calculated from the kinetic rate constants.
^{c}Association rate constants calculated using k_{d} = 2E−4 s⁻¹ for Mab5A10 and 8E−5 s⁻¹ for the nanopartide-antibody bioconjugates.

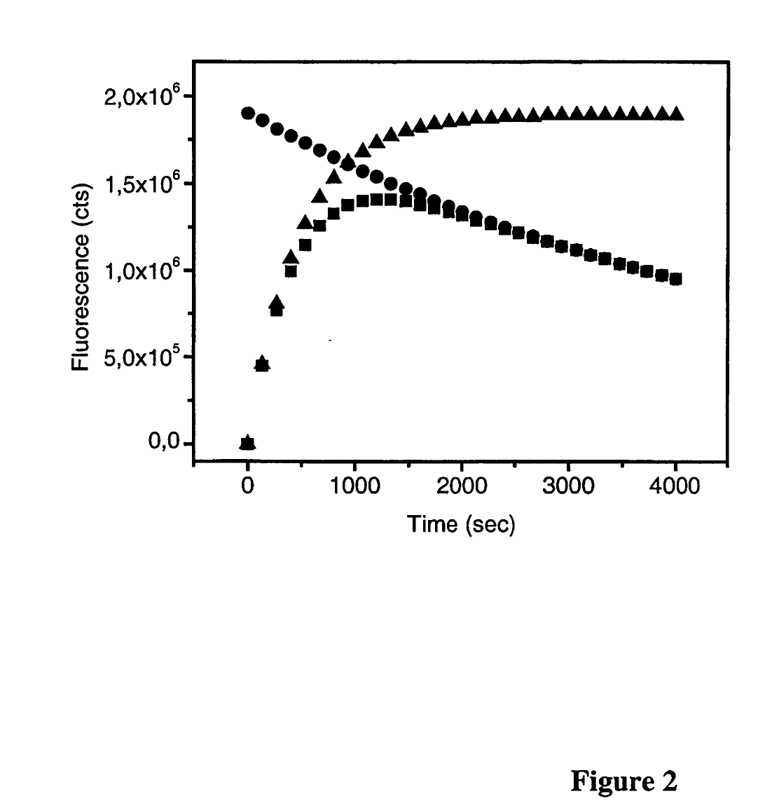

FIGS. 2 to 14 exemplify assays according to the invention as well as demonstrate the features of these assays.

FIG. 2 shows a simulation of an assay reaction where analyte and a second biospecific binding reactant react with a first solid-phase surface-bound capture biospecific binding reactant and thereafter the nanoparticles coated with a third biospecific binding reactant react with the second biospecific binding reactant: apparent curve (▪), the association of the nanoparticles with the second biospecific binding reactant (▴) and the dissociation of the second biospecific binding reactant from the analyte bound on the surface ().

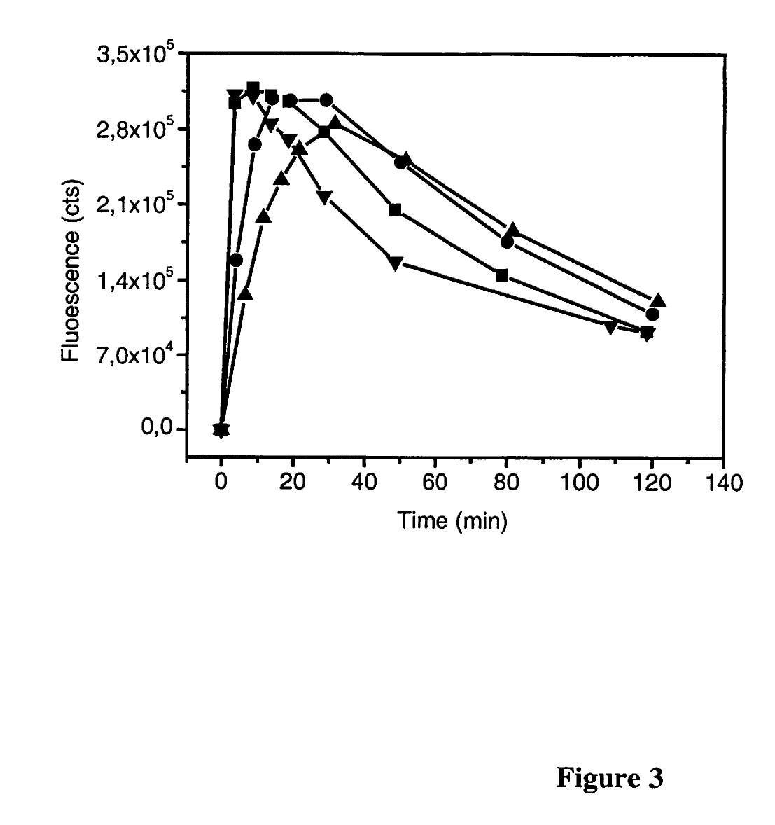

FIG. 3 shows kinetic curves of PSA assays where PSA (5 μl, 1 μg l⁻¹) and a biotinylated anti-PSA antibody 5A10 (0.6 nmol l⁻¹) reacted with a microtiter well surface-bound anti-PSA antibody H117 in a total volume of 30 μl for 15 min and thereafter the nanoparticles coated with streptavidin reacted with the biotinylated anti-PSA antibody 5A10 in a total volume of 40 μl. The curves represent the time dependent reaction of streptavidin-coated nanoparticles with the biotinylated anti-PSA 5A10 antibody bound to analyte bound to the surface-captured anti-PSA antibody H117. The number of streptavidin-coated nanoparticles was varied: 3.5·10⁸ (▴), 5·10⁸ (), 1·10⁹ (▪), and 3·10⁹ (▾) nanoparticles per reaction. A Victor 1420 (Perkin Elmer Life Sciences, Wallac Oy) time-resolved fluorometer was used to detect PSA directly on the surface of the microtiter well.

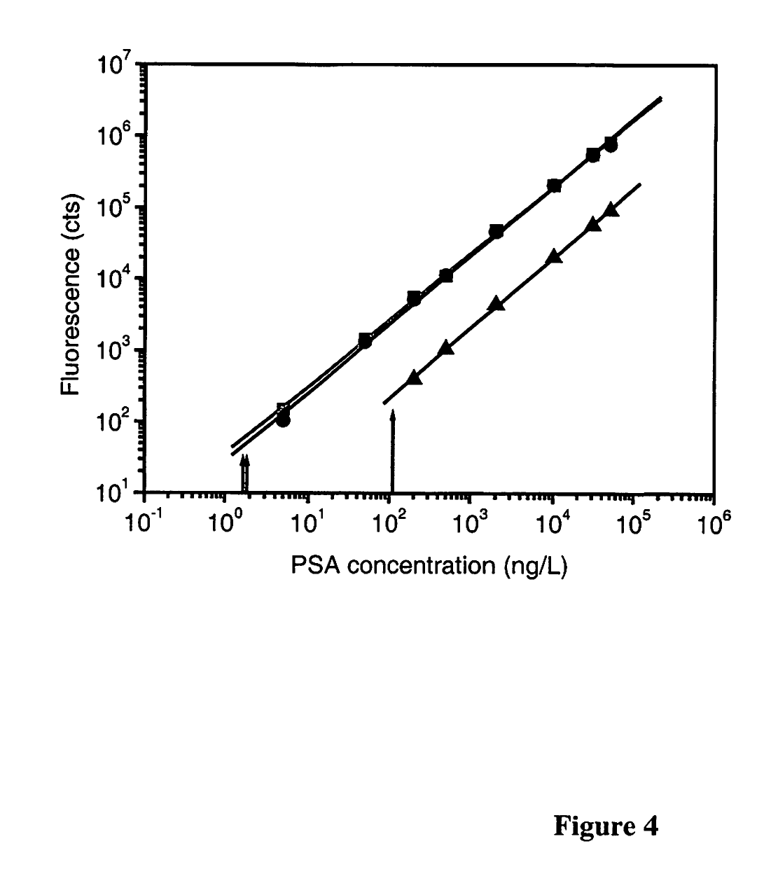

FIG. 4 shows calibration curves of PSA assays where PSA (5 μl) and a biotinylated anti-PSA antibody (0.6 nmol l⁻¹) reacted with a microtiter well surface-bound anti-PSA antibody in a total volume of 30 μl for 15 min and thereafter after a wash step 3·10⁹ Eu(III)-labeled nanoparticles coated with streptavidin (▪) or 5·10¹¹ of Eu(III)-labeled streptavidin (Eu(III)-N1-ITC chelate, Perkin Elmer Life Sciences, Wallac Oy) (▴) reacted or thereafter without the wash step 3·10⁹ Eu(III)-labeled nanoparticles coated with streptavidin () reacted with the biotinylated anti-PSA antibody in a total volume of 40 μl for 5 min. After the Eu(III)-labeled streptavidin incubation, Eu(III) ions were dissociated from the chelate to a commercial enhancement solution (Perkin Elmer Life Sciences, Wallac Oy). A Victor 1420 (Perkin Elmer Life Sciences, Wallac Oy) time-resolved fluorometer was used to detect the PSA-bound streptavidin nanoparticles directly on the surface of the microtiter well and PSA-bound Eu(III)-labeled streptavidin in solution.

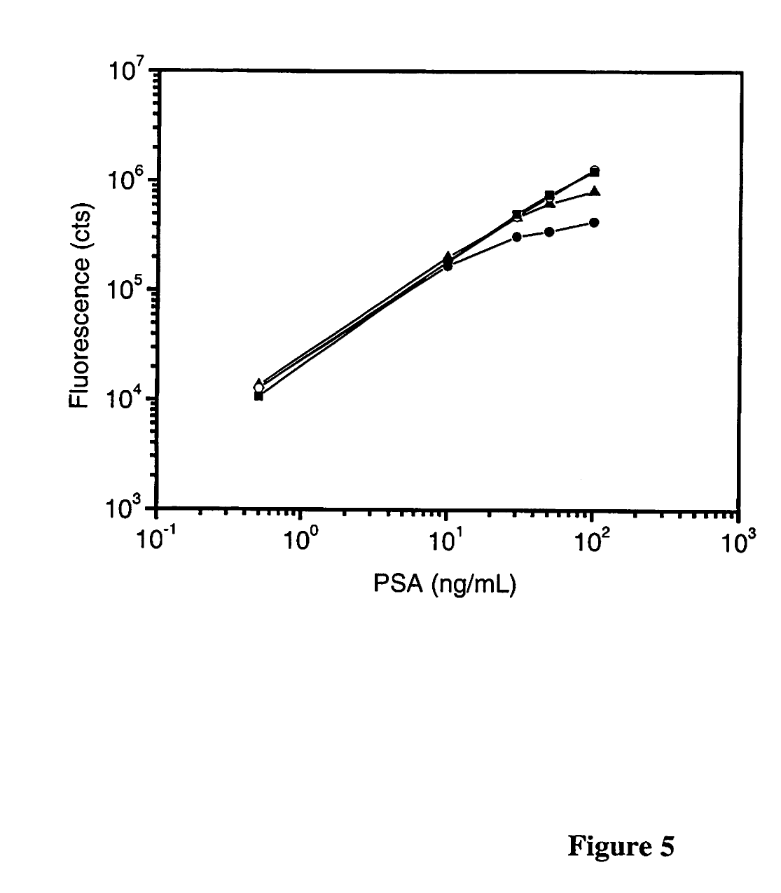

FIG. 5 shows calibration curves of PSA assays where PSA (5 μl) and a biotinylated anti-PSA antibody (0.6 nmol l⁻¹) reacted with a microtiter well surface-bound anti-PSA antibody in a total volume of 30 μl for 15 min and thereafter Eu(III)-labeled nanoparticles coated with streptavidin reacted with the biotinylated anti-PSA antibody in a total volume of 40 μl for 6 min. The number of streptavidin-coated nanoparticles was varied: 5·10⁸ (), 1 ·10⁹ (▴), 3 ·10⁹ (O), and 6·10⁹ (▪) nanoparticles per reaction. A Victor 1420 (Perkin Elmer Life Sciences, Wallac Oy) time-resolved fluorometer was used to detect the PSA-bound streptavidin nanoparticles directly on the surface of the microtiter well.

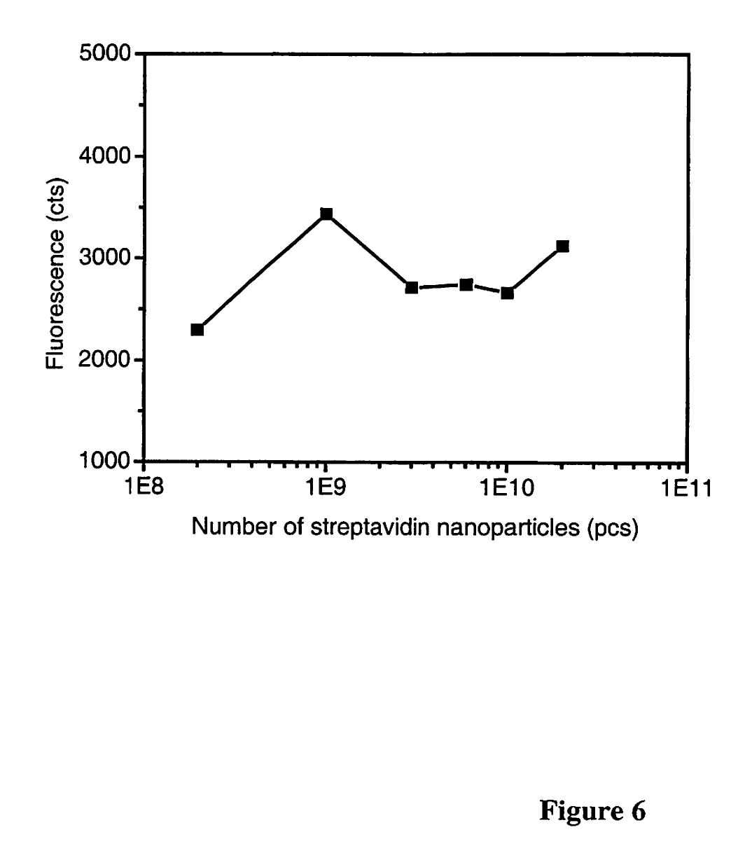

FIG. 6 shows zero concentration level signals of PSA (5 μl) vs. the number of streptavidin nanoparticles in the assays where a biotinylated anti-PSA antibody (0.6 nmol l⁻¹) reacted with a microtiter well surface-bound anti-PSA antibody non-specifically in a total volume of 30 μl for 10 min and thereafter Eu(III)-labeled nanoparticles coated with streptavidin reacted with the biotinylated anti-PSA antibody and microtiter well surface-bound anti-PSA antibody non-specifically in a total volume of 40 μl for 10 min. A Victor 1420 (Perkin Elmer Life Sciences, Wallac Oy) time-resolved fluorometer was used to detect the streptavidin nanoparticles directly on the surface of the microtiter well.

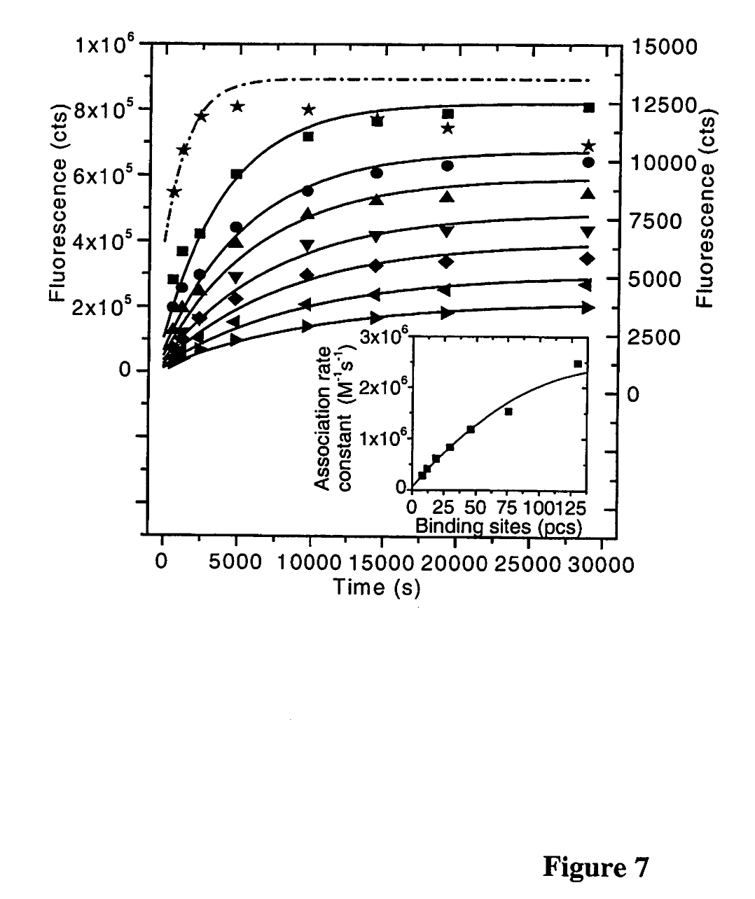

FIG. 7 shows determination of association rate constants of the Eu(III) labeled anti-PSA antibody (asterisk) and Eu(III)-labeled nanoparticle-antibody bio-conjugates with 130 (square), 76 (circle), 46 (triangle pointing up), 30 (triangle pointing down), 19 (diamond), 12 (triangle pointing left) and 8 (triangle pointing right) active binding sites. Solid lines are fitted curves for the bioconjugates using constant k_{d}=8·10⁻⁵ s⁻¹ and dashed line is for the antibody based on the first four data points and k_{d}=2 ·10⁴ s⁻¹. The number of analyte molecules were adjusted so that only a few percent of nanoparticle-antibody bioconjugates or labeled antibodies were bound, allowing a fixed value for free bioconjugate and antibody concentration. First, 5 μl of blanks or free PSA standards (0.5 μg l⁻¹ for bioconjugates, 2.5 μg l⁻¹ (for antibody) and 25 μl/well of assay buffer were added to anti-PSA antibody coated microtiter wells. The wells were incubated for 45 min and washed before 1.5 ·10⁹ pcs/well nanoparticle-antibody bioconjugates or 2 ng/well labeled antibody were added to 40 μl/well of assay buffer. Separate wells were incubated for 10-480 min and washed before measurement of the surface bound nanoparticle or antibody fraction. Time-resolved europium(III) fluorescence from the nanoparticle-antibody bioconjugates was detected directly from the bottom of the well using a Victor™ 1420 fluorometer, at 613 nm with a narrow emission aperture and lowered focus level. Fluorescence from the europium(III) labeled antibody was measured in the DELFIA® enhancement solution, at 613 nm. The fluorescence signals obtained after subtracting the non-specific binding from the total binding were plotted by fluorescence vs. time. Separate fluorescence signals, equal to the total analyte concentration (Rₘₐₓ) for nanoparticle-antibody bioconjugates and antibodies, were employed in fitting the experimental data to an integrated form of the kinetic rate equation (O'Shannessy D J et al. Anal. Biochem. 1993, 212, 457-68):

Rₜ=CkₐRₘₐₓ{1−exp{−(Ckₐ+k_{d})t}}/(Ckₐ+k_{d})

In the equation

C=bioconjugate or antibody concentration (M),

Rₜ=fluorescence signal (cts),

t=time (s),

kₐ=association rate constant (M⁻¹ s⁻¹) and k_{d}=dissociation rate constant (s⁻¹).

The inset shows the dependence of the fitted association rate constants and the number of binding sites on the bioconjugates. The calculated association rate constant for the antibody was 1.3 ·10⁶ M⁻¹ s⁻¹. The error bars reflect the ±SD of three replicas.

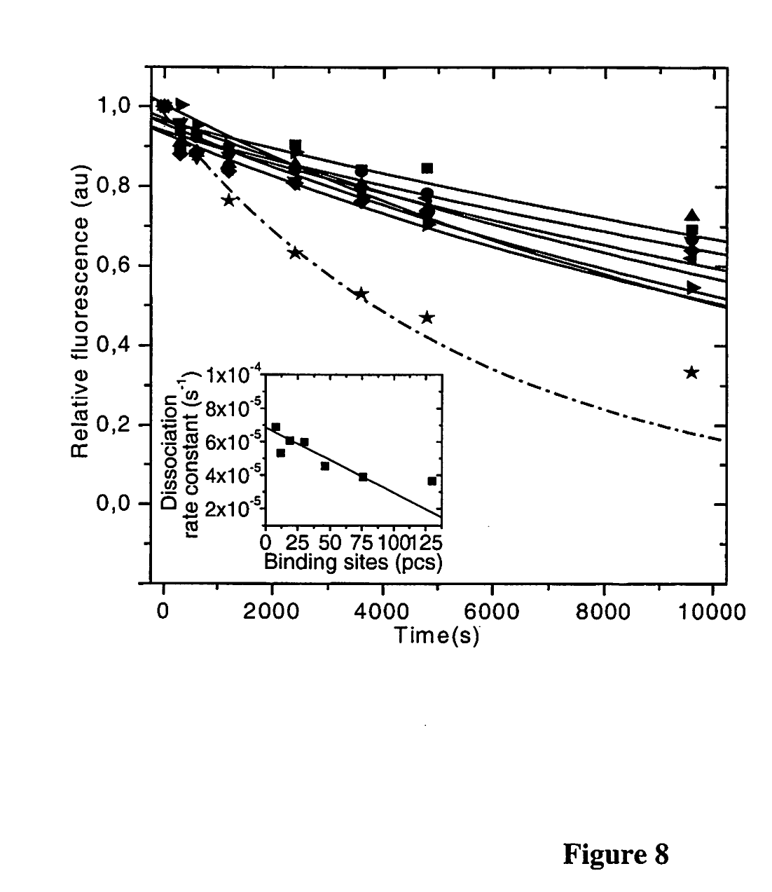

FIG. 8 shows dissociation kinetics for the Eu(III) labeled anti-PSA antibody (asterisk) and the Eu(III) labeled nanoparticle-antibody bioconjugates with 130 (square), 76 (circle), 46 (triangle pointing up), 30 (triangle pointing down), 19 (diamond), 12 (triangle pointing left) and 8 (triangle pointing right) active binding sites. The relative background subtracted fluorescence is plotted as a function of time and the lines represent dissociation calculated from determined rate constants. The last time points were discarded from rate constant determination. The calculated dissociation rate constant for the antibody is 1.8 ·10⁻⁴ s⁻¹. First, 5 μl of blanks or free PSA standards (0.5 μg l⁻¹ for bioconjugates, 48 μg l⁻¹ for antibody) and 25 μl/well of assay buffer were added to anti-PSA antibody coated microtiter wells. The wells were incubated for 45 min and washed before 1.5·10⁹ pcs/well nanoparticle-antibody bioconjugates or 75 ng/well labeled antibody were added to 40 μl/well of assay buffer. The wells were incubated for 2 h and washed. The dissociation was initiated by adding 800 ng/well of non-labeled antibody in assay buffer (50 μl/well). The wells were incubated for 0-160 min and washed, before the measurement of the surface bound nanoparticle or antibody fraction. The fluorescence signals obtained after subtracting the non-specific binding from the total binding were plotted by ln(fluorescence(time=0)/fluorescence(time)) vs. time. The slope represented the dissociation rate constant of pure dissociation phase reaction, based on an integrated form of the rate equation ln(R₀/Rₙ)=k_{d}(tₙ−t₀), where R₀ was a response at time t₀ and Rₙ response at tₙ. The inset shows the dependence of the fitted dissociation rate constants of the number of binding sites on the nanoparticle-antibody bioconjugates. The error bars reflect the ±SD of three replicas.

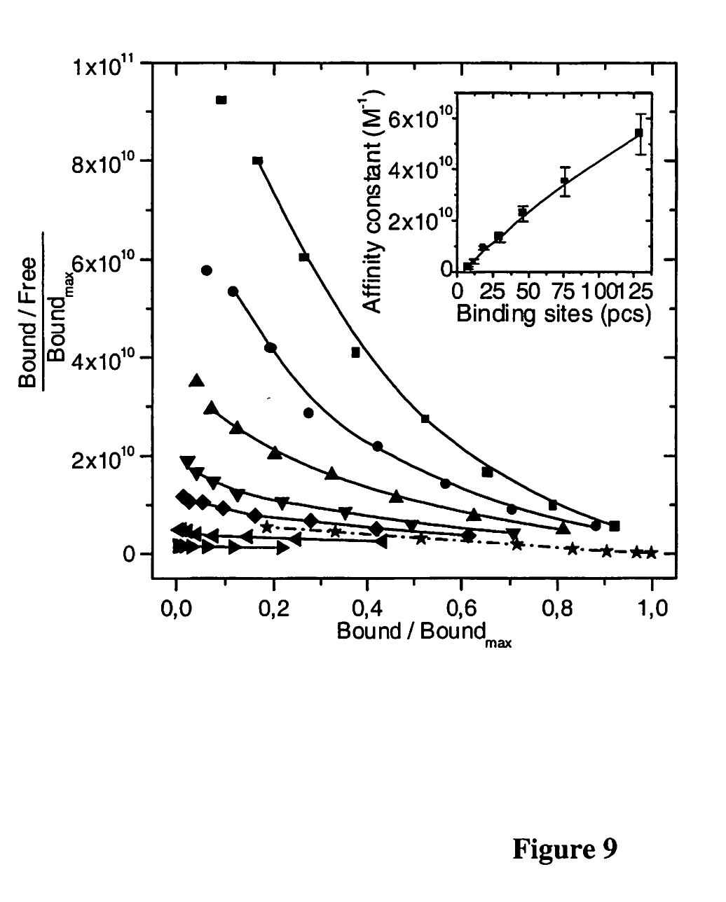

FIG. 9 shows affinity determination of the Eu(III) labeled anti-PSA antibody (asterisk) and the Eu(III) labeled nanoparticle-antibody bioconjugates with 130 (square), 76 (circle), 46 (triangle pointing up), 30 (triangle pointing down), 19 (diamond), 12 (triangle pointing left) and 8 (triangle pointing right) active binding sites. The background subtracted data is plotted to normalized Scatchard presentation enabling the direct comparison of affinities. First, 5 μl of blanks or free PSA standards (1.0 μg l⁻¹ for bioconjugates, 48 μg l⁻¹ for antibody) and 25 μl/well of assay buffer were added to anti-PSA antibody coated microtiter wells. The wells were incubated for 45 min and washed before eight concentrations, 4.0·10⁹-3.1 ·10⁷ pcs/well nanoparticle-antibody bioconjugates or 50-0.39 ng/well labeled antibody were added to 40 μl/well of assay buffer to different wells. Separate blanks were included for each nanoparticle and antibody concentration. Wells were incubated for 16 h or 4 h, for nanoparticle and antibody strips, respectively, and washed, before the measurement of the surface bound bioconjugate or antibody fraction. The bound concentrations of bioconjugates and antibodies were calculated from the fluorescence signals obtained after subtracting the non-specific binding from the total binding. Affinity constants for nanoparticle-antibody bioconjugates and labeled antibody were calculated using data plotted by bound/free ratio vs. bound concentration. The inset shows the dependence of the fitted affinity constants and the number of binding sites on the nanoparticle-antibody bioconjugates. The four data points with the lowest bound/boundₘₐₓ ratios were omitted from each linear regression analysis to obtain a value representing better monovalent binding affinity. The calculated affinity constant for the antibody was 6.6·10⁹ M⁻¹. The error bars reflect the ±SD of three replicas.

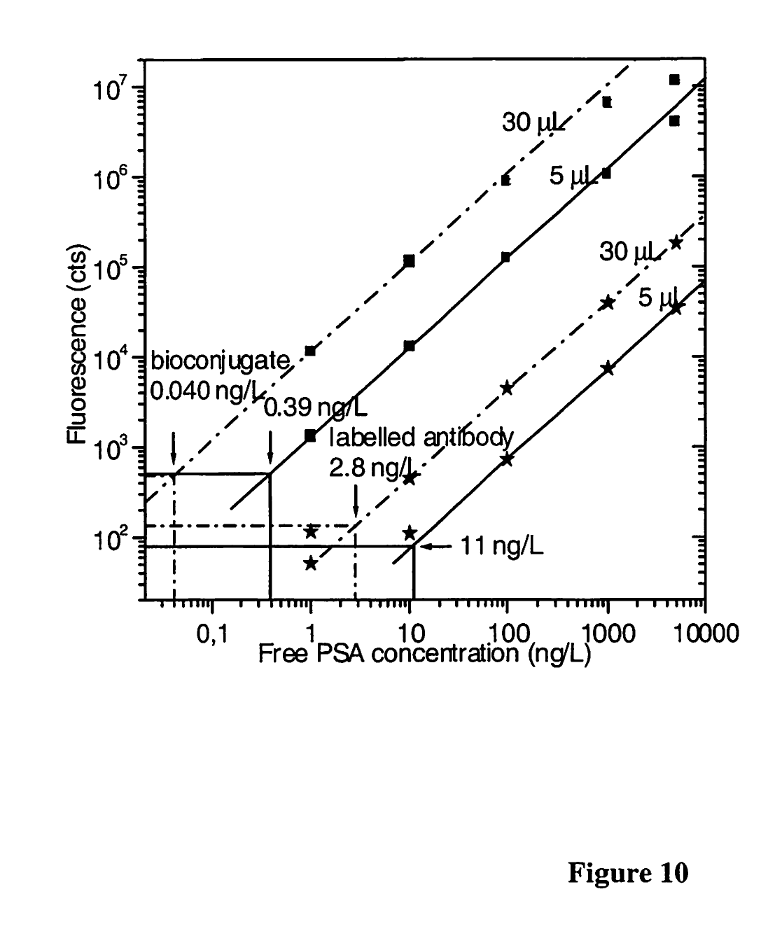

FIG. 10 shows standard curves for bioconjugate (214 active binding sites, square) and labeled antibody (8 europium(III) ions per antibody, asterisk) based two-step, non-competitive immunoassays of free PSA using 5 μl (solid line) and 30 μl (broken line) of sample. The labeled horizontal and vertical lines represent 2×SD of the blank sample and the analytical sensitivity of the assay, respectively. The solid lines are for 30 μl and broken lines for 5 μl of sample, the upper lines for bioconjugate and the lower lines for labeled antibody. First, 5 μl/well of standard and 25 μl/well of the assay buffer or only 30 μl/well standard were added into anti-PSA microtiter well and incubated for 45 min before the wells were washed. Subsequently, 1 ·10⁹ pcs/well of Eu(III) labeled nanoparticle-antibody bioconjugates or 75 ng/well of labeled antibody was added in 40 μl/well volume of assay buffer. Incubation was carried out for 2 h and the wells were washed before measurement of surface-bound fluorescence. Europium(III) fluorescence from the nanoparticle-antibody bioconjugates was detected as in the section non-specific binding but with damped emission aperture. The signal from the labeled antibody was measured at 613 nm with standard protocol after an additional incubation with 200 μl/well of the DELFIA® enhancement solution. The absolute specific signals cannot be directly compared between the bioconjugate and the labeled antibody since the nanoparticle associated fluorescence is measured from the surface with damped emission aperture. The error bars reflect the ±SD of three replicas.

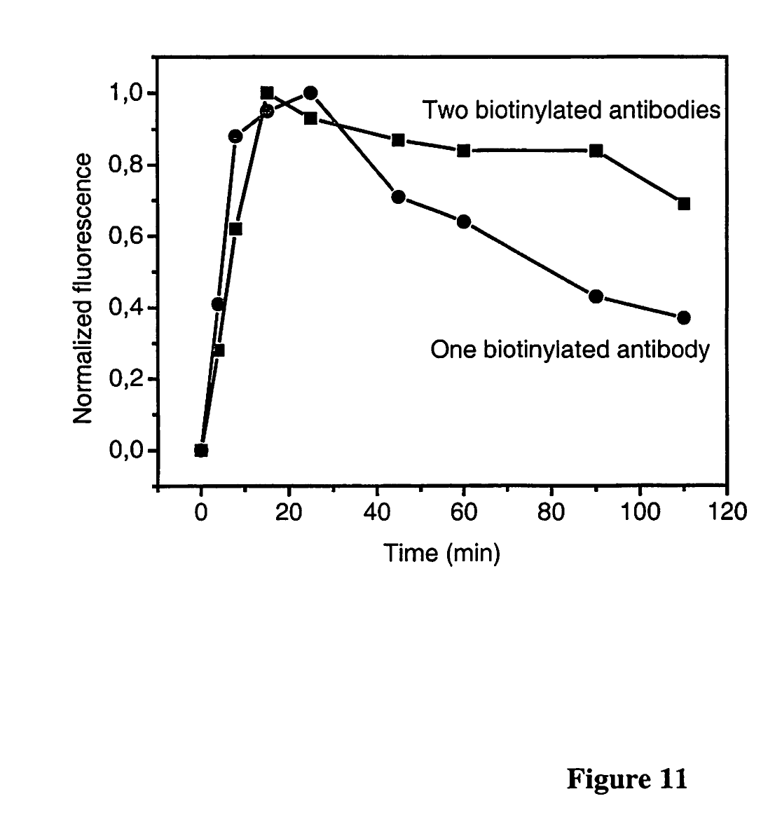

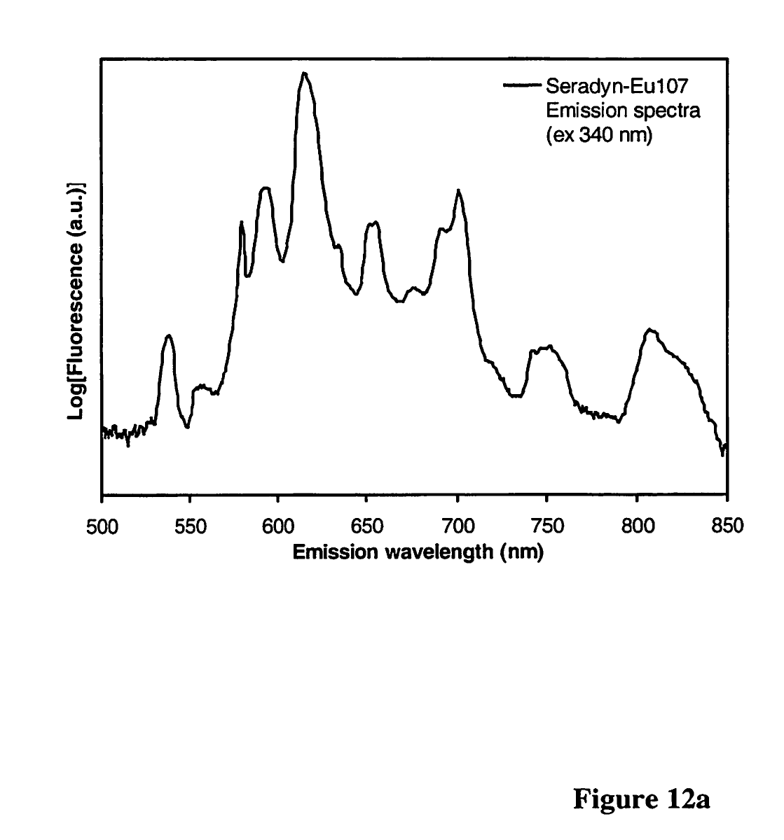

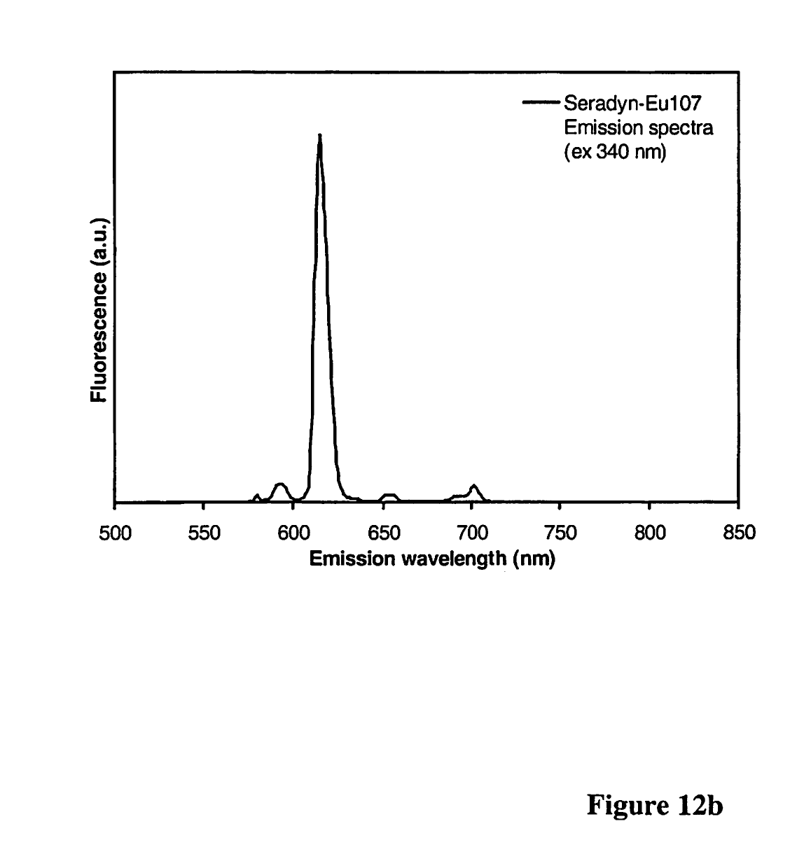

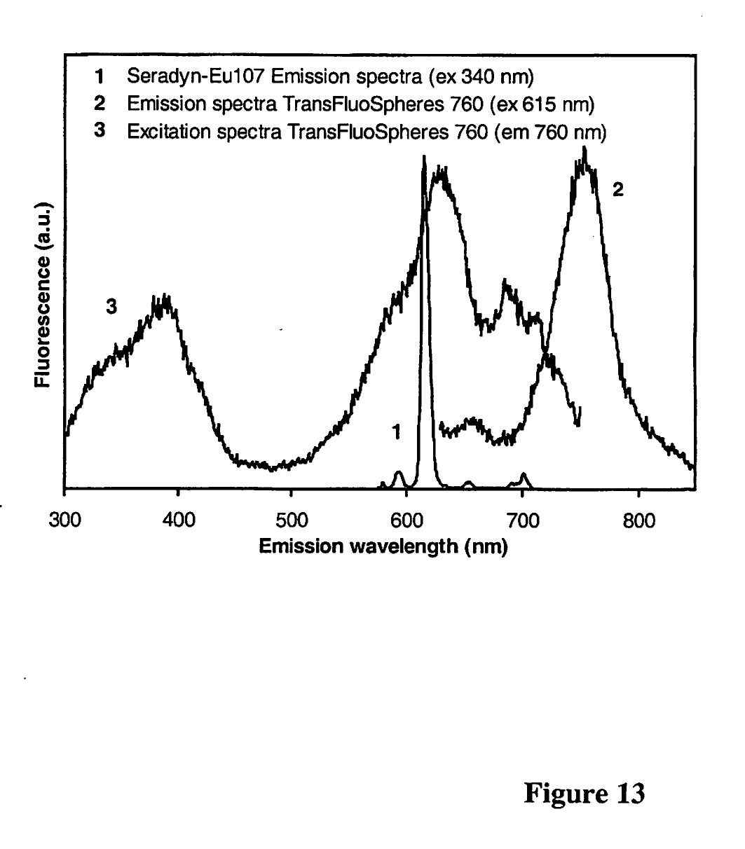

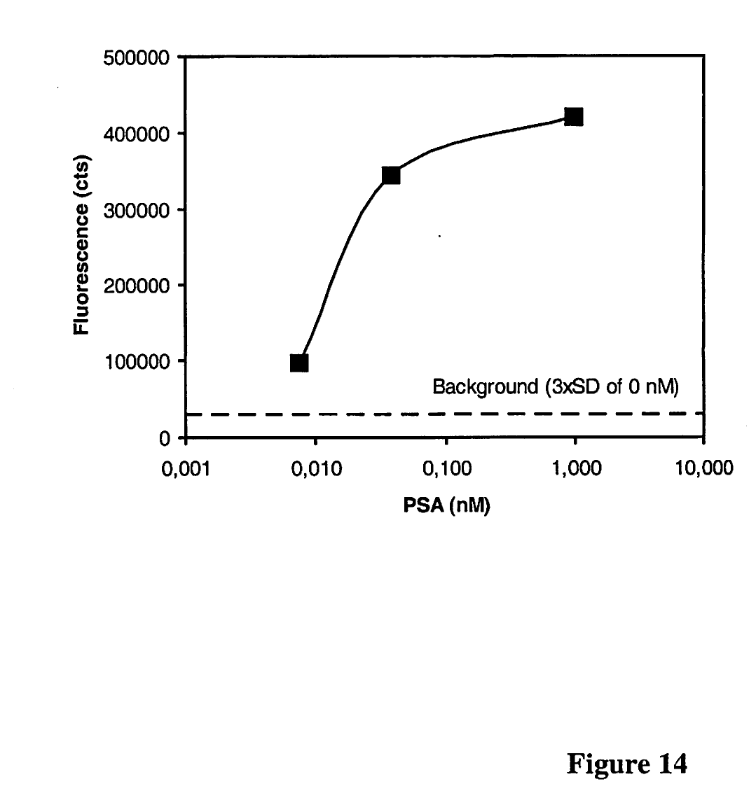

FIG. 11 shows kinetic curves of PSA assays where PSA (5 μl, 1 μg l⁻¹) and a biotinylated anti-PSA antibody 5A10 (0.6 nmol () or PSA (5 μl, 1 μg l⁻¹) and a biotinylated anti-PSA antibody 5A10 (0.6 nmol l⁻¹) and a biotinylated anti-PSA antibody H50 (0.6 nmol l⁻¹) (▪) both reacting on different sites of PSA molecule reacted with a microtiter well surface-bound anti-PSA antibody in a total volume of 30 μl for 15 min and thereafter 1 ·10⁹ nanoparticles coated with streptavidin reacted with the biotinylated anti-PSA antibodies in a total volume of 40 μl. The curves represent the time dependent reaction of streptavidin-coated nanoparticles with the biotinylated anti-PSA 5A10 antibodies (two antibodies per PSA molecule) bound to a analyte bound to the surface-captured anti-PSA antibody H117. A Victor 1420 (Perkin Elmer Life Sciences, Wallac Oy) time-resolved fluorometer was used to detect PSA directly on the surface of the microtiter well.

FIG. 12a (logarithmic) and 12b (linear scale) shows time-resolved emission spectrum of europium chelate containing fluorescent latex (Fluoro-Max, diameter 10⁷ nm, Seradyn, IN). The inlet of 12a shows precise emission profile above 700 nm. Only insignificant (cannot be distinguished from the background) direct, long lifetime emission of europium exists above 710 nm. Europium(III) fluorescence of 0.1% nanoparticle solution in 0.1% Triton X-100 was measured (Hamamatsu P M T R2949) in time-resolved fluorescence mode 340 nm excitation, 150 ms delay after excitation flash and 500 ms measurement window.

FIG. 13 shows excitation and emission spectra of multiple dye containing (energy transfer) microparticles (Transfluorespheres 760, TFS-760, diameter 2 mm, Molecular Probes, Nederlands) and emission spectra of europium chelate containing fluorescent latex (Fluoro-Max, diameter 10⁷ nm, Seradyn, IN). TFS-760 particles have exceptionally large Stoke's shift, difference between excitation and emission wavelengths. Background is too low to be shown on linear scale. Fluorescence from solution containing 40′000 particles/ml in 0.1% Triton X-100 was measured (Hamamatsu P M T R2949) in (prompt) fluorescence mode using excitation wavelength of 615 nm for emission spectra and emission wavelength of 760 nm for excitation spectra. TFS-760 particles can be efficiently excited at the wavelength of emission maximum of europium(III) luminescence and they have strong emission at 760 nm were europium(III) has a very weak background. Temporal resolution is required to separate energy transfer excited emission since, TransFluoSpheres are also excited at the excitatation wavelength of europium nanoparticles (340 nm).

FIG. 14 shows a calibration curve of a real homogeneous immunoassay of free prostate specific antigen (PSA). A non-competitive sandwich immunoassay was performed using europium chelate containing fluorescent latex (Fluoro-Max, diameter 107 nm, Seradyn, IN) as energy donor, coated with the first antibody of the sandwich-pair (Mab5A10), and multiple dye containing (energy transfer) microparticles (Transfluorespheres 760, diameter 2 mm, Molecular Probes, Nederlands) as energy acceptor, coated with the second antibody of sandwich pair (MabH117). Detection limit below 0.01 nM of free PSA in solution was achieved in the experiment using non-optimized measurement instrument. Europium(III) nanoparticles coated with first antibody (5 ·10⁹ pcs) and TFS-760 particles coated with second antibody (1.4 ·10⁹ pcs) were added in 150 mL total volume of assay buffer (50 mM Tris-HCl, pH 7.8, containing 0.9 w/v % NaCl, 0.05 w/v % NaN3, 0.5 w/v % bovine serum albumin, 0.01 w/v % Tween 40, 0.05 w/v % bovine g-globulin, 20 mM DTPA) to mictotiter well coated with bovine serum albumin to block well surface from non-specific binding. Subsequently, 50 mL of different PSA standards were added to separate wells and reaction was incubated for 2 h in room temperature with shaking. Energy transfer excited fluorescence of TFS-760 was measured using Victor multilabed counter in time-resolved mode (excitation at 340 nm, delay time 80 ms, measurement window 500 ms, cycle time 4000 ms) equipped with red-sensitive PMT and longpass emission filter (>725 nm, T % 50 at 725 nm, T % 5 at 710 nm).

1-28. (canceled)

29. A nanoparticle comprising a specific binding reactant, said nanoparticle being useful for determining an analyte, or complex comprising said analyte, to which said binding reactant is specific, wherein
a) the diameter of said nanoparticle is less than 120 nm,
b) said nanoparticle is coated with a sufficient number of specific binding reactants such that
i) the monovalent affinity constant of said nanoparticle towards said analyte exceeds that of free said binding reactant towards said analyte, and/or
ii) the monovalent association rate constant between said nanoparticle and said analyte exceeds the monovalent association rate constant between free said binding reactant and said analyte; and
c) said nanoparticle comprises a detectable feature.

30. The nanoparticle of claim 29, wherein said binding reactant is selected from the group consisting of an antibody, an antigen, a receptor ligand, a specific binding protein, avidin, streptavidin, biotin, a nucleic acid, a peptide, a sugar, a hapten, a virus, a bacteria and a cell.

31. The nanoparticle of claim 29, wherein said detectable feature is a luminescent label.

32. The nanoparticle of claim 31, wherein said luminescent label is selected from the group consisting of time-resolved fluorescent labels, upconverting fluorescent labels, rapidly decaying fluorescent labels, chemiluminescent labels and bioluminescent labels.

33. The nanoparticle of claim 29, wherein said specific binding reactant is attached to said nanoparticle by a means selected from the group consisting of adsorption, covalent coupling, grafting, solid phase synthesis, and another specific binding reactant.
